SEQUENCE LISTING (6 sequences)

SEQ ID NO: 1 - DNA, 5537 bp, Homo sapiens
aggcagaagccggcggcgcgcggacagccagtcggcgccgcgcggagctggccgctggattggctgcaacactcgcgtgtcaggcggttgctaggctccggccgcgcgccccgcccttgcgctcagcgccctctcaccgcccggtacgtgctcgcgcgaaggctgcggcgcggcgctcgcgcctcttaggcttggcggtggcggcggcggcagcttcgcgccgaatccccggggagcggcggtggcggcgtcctggggccaggaggagcgaacacctgccgcggtcctcccgccggcgctgggctctgtgtgctccgggatggagcaggtgtgcagagggtgagaacccagctctgggaccaagtcacttgcttccttacttagcaagactatcgacttgagcaaacttggacctgggatgaggatgtctgtgggcctctcgctgctgctgcccctctgggggaggacctttctcctcctgctctctgtggttatggctcagtcccactggcccagtgaaccctcagaggctgtcagggactgggaaaaccagcttgaggcatccatgcactcagtgctctcagacctccacgaggctgttcccacagtggttggcattcctgatggcacggctgtcgtcgggcgctcatttcgagtgaccattccaacagatttgattgcctccagtggagatatcatcaaggtatcagcggcagggaaggaggctttgccatcttggctgcactgggactcacagagccacaccctggagggcctcccccttgacactgataagggtgtgcattacatttcagtgagcgctacacggctgggggccaacgggagccacatcccccagacctccagtgtgttctccatcgaggtctaccctgaagaccacagtgagctgcagtcggtgaggacagcctccccagaccctggtgaggtggtatcatctgcctgtgctgcggatgaacctgtgactgttttgacggtgattttggatgccgacctcaccaagatgaccccaaagcaaaggattgacctcctgcacaggatgcggagcttctcagaagtagagcttcacaacatgaaattagtgccggtggtgaataacagactatttgacatgtcggccttcatggctggcccgggaaatgcaaaaaaggtggtggagaatggggcccttctctcctggaagctgggctgctccctgaaccagaacagtgtgcctgacattcatggtgtagaggcccctgccagggagggcgcaatgtctgctcagcttggctaccctgtggtgggttggcacatcgccaataagaagccccctcttcccaaacgcgtccggaggcagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctccatcgtggtggaatggaccaacaacacactgcccttggagccctgccccaaggagcagatcgctgggctgagccgccggatcgctgaggatgatggaaaacctcggcctgccttctccaacgccctagagcctgactttaaggccacaagcatcactgtgacgggctctggcagttgtcggcacctacagtttatccctgtggtaccacccaggagagtgccctcagaggcgccgcccacagaagtgcctgacagggaccctgagaagagcagtgaggatgatgtctacctgcacacagtcattccggccgtggtggtcgcagccatcctgctcattgctggcatcattgccatgatctgctaccgcaagaagcggaagggcaagcttacccttgaggaccaggccaccttcatcaagaagggggtgcctatcatctttgcagacgaactggacgactccaagcccccaccctcctccagcatgccactcattctgcaggaggagaaggctcccctaccccctcctgagtaccccaaccagagtgtgcccgagaccactcctctgaaccaggacaccatgggagagtacacgcccctgcgggatgaggatcccaatgcgcctccctaccagcccccaccgcccttcacagcacccatggagggcaagggctcccgtcccaagaacatgaccccataccggtcacctcctccctatgtcccaccttaacccgcaagcgcctgggtggaggcagggtagggcaggggcctggagacgacatggtgttgtctgtggagaccggtggcctgcagaccattgcccaccgggagccgacacctgacctagcacacactgacacaggggcctggacaagcccgccctctctggtcctcccaaaccccaaagcagctggagagactttggggacttttttatttttattttttgcctaacagcttttggtttgttcatagagaattcttcgcttcatttttgatggctggctctgaaagcaccatgtggagtggaggtggagggagcgaggaaccatgaatgaactcgcaggcagtgccgggcggccccctggctctctgcgttttgcctttaacactaactgtactgttttttctattcacgtgtgtctagctgcaggatgtaacatggaaaacagtaactaaagattaaattcaaaggactttcagaagttaaggttaagtttttacgtttaatctgctgtttacctaaacttgtatgtataatttttgggtgggtatggggaattgctttgctaaaaataagctcccagggtgtttcaaacttagagaagaccaagggacagtattttttatcaaaggaatactattttttcacactacgtcaacttggttgctctgataccccagagcctgattgggggcctcccggccctggctcacgccaagtccctggtgctgggtttgctctcccgctgttgccaggggctggaagctggaggggtctcttgggccatggacatccccacttccagcccatgtacactagtggcccacgaccaaggggtcttcatttccatgaaaaagggactccaagaggcagtggtggctgtggcccccaactttggtgctccagggtgggccagctgcttgtgggggcacctgggaggtcaaaggtctccaccacatcaacctattttgttttaccctttttctgtgcattgtttttttttttcctcctaaaaggaatatcacggttttttgaaacactcagtgggggacattttggtgaagatgcaatatttttatgtcatgtgatgctctttcctcacttgaccttggccgctttgtcctaacagtccacagtcctgccccgacccaccccatcccttttctctggcactccagtcccaggccttgggcctgaactactggaaaaggtctggcggctggggaggagtgccagcaatagttcataataaaaatctgttagctctcaaagctaattttttactaaagtttttatacagcctcaaattgttttattaaaaaaaagatttaaaatggtgatgcttacagcagtttgtacgagctcttaagtgttgattccatggaactgacggctttgcttgttttgattcttttccccctacttttcctaatggtttaaattctggaattacactggggttcttttgccttttttagcagaacatccgtccgtccatctgcatctctgtcccatgactcaggggcgcccactctgcttcgattctcctcctgtggaagaaaccattttgagcatgacttttcttgatgtctgaagcgttattttgggtactttttagggaggaatgcctttcgcaataatgtatccattccctgattgagggtgggtgggtggacccaggctccctttgcacacagagcagctacttctaagccatatcgactgttttgcagaggatttgtgtgtgctgcctcaggaggggagggctggtaggagggggggagaggtctctgtcctactgctctccagagggcatttccccttgcgccttctcccacagggcccagcccctctcccctgccccagtccccagggggtactctggagtgagcagtgcccctgtgggggagcctgtaaatgcgggctcagtggaccactggtgactgggctcatgcctccaagtcagagtttccctggtgccccagagacaggagcacaagtgggatctgacctggtgagattatttctgatgacctcatcaaaaaataaacaattcccaatgttccaggtgagggctttgaaaggccttccaaacagctccgtcgcccctagcaactccaccattgggcactgccatgcagagacgtggctggcccagaatggcctgttgccatagcaactggaggcgatggggcagtgaacagaataacaacagcaacaatgcctttgcaggcagcctgctcccctgagcgctgggctggtgatggtcgttggactctgtgagatggagagccaatctcacattcaagtgttcaccaaccactgatgtgtttttatttccttctatatgattttaagatgtgttttctgcattctgtaaagaaacatatcaaactaaataaaagcagtgtctttattacaaaaaaaaaaaaaaaaa

SEQ ID NO: 2 - protein, 895 aa, Homo sapiens
MRMSVGLSLLLPLWGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 3 - protein, 624 aa, Homo sapiens
HWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRG

SEQ ID NO: 4 - DNA, 2032 bp, Homo sapiens
ccggctctgggatcaagtcacttgcttgcttccttagcaagatcttcggcttgagcgaacttggcctgggatgaggatgtctgtgggcctttcactgctgctccccttgtgggggaggacatttctcctcctcctctgtgtggccgtggctcagtcccattggcccagcgaaccctcggaggctgtcagggactgggagaaccagctggaggcgtccatgcactctgtgctctcagacctgcacgaagcccttcccacagtggttggcattcctgatggcacggctgttgttgggcgctcgtttcgagtgaccattccaacagatttaattggctccagtggagaagtcatcaaggtatccacggcagggaaggaggttttgccatcgtggctgcattgggatccacagagccacaccctggagggccttccgctggacacggacaagggtgtgcattacatctcagtgagcgctgcacagctggatgccaacggaagccacatccctcagacctccagtgtgttctccatcgaggtctaccccgaagaccacagtgagccgcagtctgtgcgggcggcctctccagacctgggcgaggcggcggcgtctgcctgtgctgccgaggagccggtgaccgtcttgaccgtgattctggatgccgatctcaccaagatgactccgaagcagaggatcgacctcctgcacaggatgcagagcttctcggaggtggagctccacaacatgaagttggtgccggtggtgaataacagactgtttgatatgtctgccttcatggccggccccggaaacgccaaaaaggtggtagagaacggggccctgctctcctggaagctgggctgctccctgaaccagaacagtgtgcctgacattcgcggcgtggaggcccctgccagggagggcactatgtctgcccagcttggctaccctgtggtgggttggcacattgccaacaagaagccacctctccccaagcgtatccgaaggcagatccatgccacacccacacctgtcactgccattgggcccccaaccacggccatccaggagccgccgtccaggatcgtgcctacccccacttctccagccattgctcctcccacagagacgatggctcctccagtcagggatcctgttcctgggaagcccacggtcaccactcggactcgaggtgccattattcagaccccaaccctaggccccatccagcccactcgggtgtcagacgctggcaccgtagtttctggccagattcgtgcaacggtgaccattcctggctacgtggagcccacagcagttgccacccctcccacaactacaaccaaaaagccacgagtgtccacaccaaaaccagcaacgccttcaacggactcctcagccaccacgactcgcaggccaaccaagaagccacggacacccaggccggtgccacgggtcaccactaaagctcccatcaccaggctggagacggcctccccacctactcgtatccgcaccaccaccagcggggtgccccgcgggggagaacccaaccagcgcccagagctcaagaaccacatcgacagggtggacgcctgggtcggcacctactttgaggtgaagatcccatctgataccttctacgacaaggaggataccaccaccgacaagctcaagctgaccctgaagctgcgagagcagcagctggtgggcgagaagtcctgggtgcagttcaacagcaacagccagctcatgtatggcctgcccgacagcagccacgtgggcaaacacgagtatttcatgcatgccacagacaagggaggcctgtccgccgtggatgcctttgagatccatgtccacaagcgccctcaaggggacaaagctcctgctcgtttcaaagccaagttcgtgggtgacccagcgccagtggtgaatgacatccacaagaagattgccctggtgaagaagctggcctttgcctttggggatcgcaattgcagcaccgtcaccctgcagaacatcacccgcggctga

SEQ ID NO: 5 - DNA, 2303 bp, Homo sapiens
gaattcgcccttggatccatgctgggaatctgcagggggagacggaaattcttggctgcctcgttgagtcttctctgcatcccagccatcacctggatttacctgttttctgggagcttcgaagatggaaagcccgtgtctctgtcaccgctggagtcccaggcacacagccccaggtacacggcctccagccagcgggagcgcgagagcctggaggtgcgcatgcgcgaggtggaggaggagaaccgcgccctccgcaggcagctcagcctggcccagggccgagccccatcccatcgccgaggcaaccactccaagacctactccatggaggagggcactggagacagcgagaaccttcgggctggcatcgtggcaggcaacagctccgagtgtgggcagcagccggtcgtggagaaatgcgagacaatccacgttgctattgtctgcgccggatacaatgccagccgggatgtcgtcaccctggtcaaatccgtcctgttccatagacggaaccctctgcacttccaccttattgctgactccattgcggagcagatcctggccacgctcttccagacctggatggtgcccgctgtgcgtgtggacttctacaatgcagacgagctcaagtctgaagtttcctggatccccaataaacattactctgggatttatggtctgatgaagcttgtcctgaccaagactcttcctgccaacctggagagagtcatcgtccttgacacggatatcacctttgccactgacattgcagagctgtgggctgtgttccacaagttcaaaggtcagcaagtcctgggcttggtggagaaccagagtgactggtaccttggaaacctgtggaaaaatcaccgcccatggccagcccttggaagaggctacaacacaggggtgatcctgttacttctggataagctgcggaagatgaaatgggagcagatgtggaggctgaccgcagagagggagctcatgggcatgctctctacatccttagctgaccaggatattttcaatgccgtcatcaaacaaaaccccttccttgtgtaccagctcccctgcttctggaatgtgcagctgtcagaccacacccgctccgagcagtgctacagagacgtgtctgatctaaaggtcattcactggaactcccccaagaagctccgggtgaagaacaagcatgtggagttttttcgcaacctctacctgaccttcctggagtatgacggcaatcttctgaggcgggaactgtttggctgccccagtgaggctgatgtcaacagtgaaaacctccagaagcagctgtctgagctggacgaggacgacctgtgctatgagttccggcgagagcgcttcactgtccaccgcacccacctgtacttcctgcactacgagtatgagcctgcagcagacagcacggacgtcaccctggtcgctcagctgtccatggacaggctccagatgctggaggccatctgcaagcactgggaggggcccatcagcctggccctctacctgtcagacgccgaggcccagcagttcctccgctacgcacagggctctgaggtgcttatgagccgccacaacgtgggctaccacatcgtgtacaaggagggccagttctaccccgtgaacctgctgcgcaacgtggccatgaagcacatcagcactccctacatgttcctgtctgacattgacttcctgcccatgtatgggctctatgagtacctcaggaagtctgtcatccagctcgatcttgccaacaccaagaaagcaatgattgtccccgcgttcgagacactgcgctaccggctgtccttccccaagtcaaaagcggagttgctgtcaatgctggacatggggaccctcttcacattcaggtaccacgtctggacgaaaggccacgcacccacaaacttcgccaagtggcggaccgccaccacgccttaccgggttgagtgggaggccgattttgagccgtatgttgttgtgagacgtgactgcccggagtacgaccggaggtttgtaggctttggctggaacaaagtggctcatatcatggagctggatgtgcaggagtatgagttcattgtgctgcccaacgcctacatgatccacatgcctcatgcccccagcttcgacattaccaagttccgttccaacaagcaataccgcatctgtctcaaaaccctcaaggaagagtttcagcaggacatgtcccgccgctacggctttgctgccctgaaatatctcacagccgagaacaacagctaggtaccaagggcgaa

SEQ ID NO: 6 - DNA, 2766 bp, Homo sapiens
gaattcgaccaagtcacttgcttccttacttagcaagactatcgacttgagcaaacttggacctgggatgaggatgtctgtgggcctctcgctgctgctgcccctctgggggaggacctttctcctcctgctctctgtggttatggctcagtcccactggcccagtgaaccctcagaggctgtcagggactgggaaaaccagcttgaggcatccatgcactcagtgctctcagacctccacgaggctgttcccacagtggttggcattcctgatggcacggctgtcgtcgggcgctcatttcgagtgaccattccaacagatttgattgcctccagtggagatatcatcaaggtatcagcggcagggaaggaggctttgccatcttggctgcactgggactcacagagccacaccctggagggcctcccccttgacactgataagggtgtgcattacatttcagtgagcgctacacggctgggggccaacgggagccacatcccccagacctccagtgtgttctccatcgaggtctaccctgaagaccacagtgagctgcagtcggtgaggacagcctccccagaccctggtgaggtggtatcatctgcctgtgctgcggatgaacctgtgactgttttgacggtgattttggatgccgacctcaccaagatgaccccaaagcaaaggattgacctcctgcacaggatgcggagcttctcagaagtagagcttcacaacatgaaattagtgccggtggtgaataacagactatttgacatgtcggccttcatggctggcccgggaaatgcaaaaaaggtggtggagaatggggcccttctctcctggaagctgggctgctccctgaaccagaacagtgtgcctgacattcatggtgtagaggcacctgccagggagggcgcaatgtctgctcagcttggctaccctgtggtgggttggcacatcgccaataagaagccccctcttcccaaacgcgtccggaggcagatccatgctacacccacacctgtcactgccattgggcccccaaccacggctatccaggagcccccatccaggatcgtgccaacccccacatctccagccattgctcctccaacgagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctccatcgtggtggaatggaccaacaacacactgcccttggagccctgccccaaggagcagatcgctgggctgagccgccggatcgctgaggatgatggaaaacctcggcctgccttctccaacgccctagagcctgactttaaggccacaagcatcactgtgacgggctctggcagttgtcggcacctacagtttatccctgtggtaccacccaggagagtgccccagaggcgccgcccacagaagtgcctgacagggaccctgagaagagcagtgaggatgatgtctacctgcatacagtcattccggccgtggtggtcgcagccatcctgctcattgctggcatcattgccatgatctgctaccgcaagaagcggaagggcaagcttacccttgaggaccaggccaccttcatcaagaagggggtgcctatcatctttgcagacgaactggacgactccaagcccccaccctcctccagcatgccactcattctgcaggaggagaaggctcccctaccccctcctgagtaccccaaccagagtgtgcccgagaccactcctctgaaccaggacaccatgggagagtacacgcccctgcgggatgaggatcccaatgcgcctccctaccagcccccaccgcccttcacagcacccatggagggcaagggctcccgtcccaagaacatgaccccataccggtcacctcctccctatgtcccaccttaagtcgagcatgcat

(19) United States
(12) Patent Application Publication
(10) Pub. No.: US 2011/0166081 A1
(43) Pub. Date: Jul. 7, 2011

(54) Alpha-dystroglycan as a Protein Therapeutic

(75) Inventors: Kevin P. Campbell, Iowa City, IA (US); Takako Moriguchi, Iowa City, IA (US)

(73) Assignee: UNIVERSITY OF IOWA RESEARCH FOUNDATION, Iowa City, IA (US)

(21) Appl. No.: 12/836,874

(22) Filed: Jul. 15, 2010

Related U.S. Application Data
(60) Provisional application No. 61/266,473, filed on Dec. 3, 2009.

(51) Int. Cl. A61K 38/17; A61P 21/00; C12P 21/02

(52) U.S. Cl. 514/20.9; 435/69.1

(57) ABSTRACT

Disclosed is alpha-dystroglycan protein (alpha-DG) in glycosylated and functional form. The disclosed alpha-DG binds to the basal lamina and to the sarcolemma of muscle fibers and may be injected into muscle and incorporated into muscle fibers in order to restore membrane integrity where the muscle fibers comprise a dysfunctional alpha-DG protein. Alpha-DG as disclosed herein may be utilized in pharmaceutical compositions and methods for treating diseases and disorders associated with or characterized by a dysfunctional alpha-DG, such as muscular dystrophy.

### CROSS-REFERENCE TO RELATED APPLICATIONS

The present application claims the benefit under 35 U.S.C. §119(e) to U.S. Provisional Application No. 61/226,473, filed on Jul. 17, 2009, the content of which is incorporated herein by reference in its entirety.

### FIELD

The field of the invention relates to alpha-dystroglycan protein (alpha-DG) and its use as a therapeutic. In particular, the field of the invention relates to the use of alpha-DG as a therapeutic for diseases and disorders associated with or characterized by a dysfunctional alpha-DG such as muscular dystrophy.

### BACKGROUND

The muscular dystrophies are genetically and clinically diverse (1, 2). Although great progress has been made in identification of genes responsible for various muscular dystrophies, the mechanistic function of these gene products and their roles in the pathogenesis of disease is not clearly understood. One reason for this lack of understanding is that primary genetic alterations often lead to secondary changes, thereby triggering multiple pathogenic pathways. Compromised integrity of the sarcolemma has been proposed as the underlying mechanism for muscular dystrophy since 1852 (3); however, the molecular basis for this mechanism has never been clearly established.

The sarcolemma of each individual skeletal muscle fiber is closely associated with an extracellular protein matrix layer—the basement membrane (4-6). This membrane comprises both an internal felt-like basal lamina and an external reticular lamina composed of at least ten secretory proteins which include members of the laminin family, perlecan, agrin, and the collagens (7, 8). The native basement membrane has a very substantial mechanical strength (5). Genetic mutations or deletions of some of these basement membrane proteins lead to a variety of defects, including early embryonic lethality and congenital muscular dystrophy. The basal lamina is linked directly to the cell membrane through transmembrane receptors including dystroglycan (DG) and the integrins, all of which bind laminin with high affinity (9, 10). In addition, alpha-DG also binds to many other basal lamina proteins containing laminin globular (LG) domains such as perlecan (11) and agrin (12). The functional role of the DG- and integrin-linked basal lamina in adult skeletal muscle physiology has not been fully investigated.

DG consists of a highly glycosylated, extracellular alpha subunit (alpha-DG) and a transmembrane beta subunit (beta-DG), both of which are encoded by the gene Dag1 and generated by post-translational cleavage and processing (13). The matrix-binding capacity of alpha-DG is dependent on its extensive post-translational glycosylation (14, 15), and this has emerged as a convergent target for a group of limb-girdle and congenital muscular dystrophies termed “secondary dystroglycanopathy.” These include Fukuyama congenital muscular dystrophy, muscle-eye-brain disease, Walker-Warburg syndrome, congenital muscular dystrophy 1C (MDC1C) and 1D (MDC1D), as well as a milder form of limb-girdle muscular dystrophy type 21. Moreover, some pathogens target properly processed alpha-DG for cellular entry, including Mycobacterium leprae, Lassa fever virus and lymphocytic choriomeningitis virus (LCMV) (16, 17). The early lethality in DG-null mice (18), the prevalence of diseases involving alpha-DG hypoglycosylation, and the co-opting of normal alpha-DG for cellular entry by pathogens, all support the hypothesis that DG-linked basal lamina plays an essential role in cell biology.

Another protein that binds laminin with high affinity, alpha7beta1 integrin, is predominantly expressed in adult skeletal muscle (10, 19). Mice lacking alpha7 integrin develop a mild form of muscular dystrophy (20) and mutations in the human integrin alpha7 gene have been found in a rare form of congenital muscular dystrophy (21). These observations suggest that the alpha7beta1 integrin complex is also important for normal skeletal muscle function. Different from alpha-DG binding to many basal lamina proteins, alpha7beta1 has only been reported to bind laminin (10).

Despite both dystroglycan and integrin alpha7 contributing to the force production of skeletal muscles, here only the disruption of dystroglycan was shown to cause detachment of the basal lamina from the sarcolemma and render muscle prone to contraction-induced injury. More specifically, disruption of the LG domain binding motif on alpha-dystroglycan is sufficient to induce these phenotypes. Using an assay that involves in situ membrane damage, sarcolemmal integrity is shown to be compromised in Large^{myd} muscles and in normal muscles when the UV-inactivated LCMV competes for association with alpha-dystroglycan. Therefore, this data suggest that the basal lamina strengthens sarcolemmal integrity and protects muscle from damage via the LG domain binding motif of alpha-dystroglycan.

In order to study the role of alpha-DG in preventing contraction-induced injury, recombinant alpha-DG protein was prepared and purified. The prepared alpha-DG was glycosylated by LARGE and observed to bind to the basal lamina and sarcolemma of muscle fibers. After being injection into muscle, the prepared alpha-DG was observed to incorporate into the muscular dystrophin-glycoprotein complex and restore membrane integrity.

### SUMMARY

Disclosed herein is a purified alpha-dystroglycan protein (alpha-DG) in glycosylated and functional form. The disclosed alpha-DG typically is glycosylated by like-acetylglucosaminyltransferase (LARGE). Functionally, the disclosed alpha-DG binds to the basal lamina and to the sarcolemma of muscle fibers and may be injected into muscle and incorporated into muscle fibers (e.g., in order to restore membrane integrity where the muscle fibers comprise a dysfunctional alpha-DG protein). The alpha-DG disclosed herein may be utilized in pharmaceutical compositions and methods for treating diseases and disorders associated with or characterized by a dysfunctional alpha-DG, such as muscular dystrophy.

As disclosed herein, alpha-DG may be formulated as a pharmaceutical composition for injection into muscle tissue. The pharmaceutical composition may comprise an effective amount of alpha-DG for treating a disease or condition associated with or characterized by a dysfunctional alpha-DG (e.g., for treating a disease or condition associated with or characterized by a dysfunctional alpha-DG that does not bind to at least one of the basal lamina and the sarcolemma of muscle fibers). In some embodiments, the pharmaceutical compositions may comprise an effective amount of alpha-DG for treating a muscular dystrophy associated with or characterized by loss of endogenous alpha-DG from a muscular dystrophin-glycoprotein complex.

The pharmaceutical compositions may comprise a purified form of alpha-DG. In some embodiments of the pharmaceutical compositions, alpha-DG represents greater than about 90% of total protein in the composition (or greater than about 95% or 99% of total protein in the composition).

The alpha-DG disclosed herein typically is glycosylated. For example, the alpha-DG disclosed herein may be glycosylated by like-acetylglucosaminyltransferase (LARGE). In some embodiment, the alpha-DG disclosed herein may be O-glycosylated, N-glycosylated, or both O-glycosylated and N-glycosylated.

The alpha-DG disclosed herein typically is mammalian. In some embodiments, the disclosed alpha-DG is human alpha-DG. The polypeptide of the alpha-DG disclosed herein may comprise an amino acid sequence of SEQ ID NO:3 and may be coded by the nucleic acid sequence of SEQ ID NO:1 or SEQ ID NO:6.

The disclosed pharmaceutical compositions comprise alpha-DG and further may comprise a pharmaceutical carrier, excipient, diluent, or stabilizer. In some embodiments, the disclosed pharmaceutical compositions further comprise a buffer. In further embodiments, the disclosed pharmaceutical compositions are sterile saline solutions. For example, the disclosed pharmaceutical compositions may be sterile and comprise about 0.80-1.00% (w/v) NaCl (or about 0.90-0.92% (w/v) NaCl).

The pharmaceutical compositions may comprise alpha-DG at any suitable concentration. In some embodiments, the pharmaceutical compositions comprise alpha-DG at a concentration of at least about 1 mg/ml (or at a concentration of at least about 10 mg/ml or at a concentration of at least about 100 mg/ml).

The disclosed pharmaceutical compositions may be utilized in methods wherein the compositions are injected into muscle tissue of a patient in need thereof. For example, the disclosed pharmaceutical composition may be injected into muscle tissue of a mammal having muscular dystrophy (e.g., a mammal having a muscular dystrophy associated with or characterized by loss of endogenous alpha-DG from a muscular dystrophin-glycoprotein complex). In the methods, after the patient is injected alpha-DG may incorporate into the muscle fibers of the patient and restore or improve membrane integrity. For example, the patient may express a dysfunctional alpha-DG that does not bind to at least one of basal lamina and the sarcolemma of muscle fibers, whereas the injected alpha-DG binds to the basal lamina and the sarcolemma of muscle fibers. Suitable patients for the disclosed methods may include human patients.

The alpha-DG disclosed herein may be in purified form and optionally may be recombinant. Methods for preparing a purified alpha-dystroglycan protein may include: (a) transfecting a cell with a vector that expresses alpha-dystroglycan protein; (b) transfecting the cell with a vector that expresses like-acetylglucosaminyltransferase (LARGE), either prior to step (a), concurrently with step (a), or after step (a); (c) culturing the transfected cell, wherein the transfected cell secretes alpha-dystroglycan protein; and (d) purifying the alpha-dystroglycan protein that is secreted from the transfected cell. Alternatively, methods for preparing a purified alpha-dystroglycan protein may include: (a) transfecting a cell that expresses alpha-dystroglycan protein with a vector that expresses like-acetylglucosaminyltransferase (LARGE); (b) culturing the transfected cell, wherein the transfected cell secretes alpha-dystroglycan protein; and (c) purifying the alpha-dystroglycan protein that is secreted from the transfected cell. In other embodiments, methods for preparing the disclosed alpha-DG may include (a) transfecting a cell with a vector that expresses like-acetylglucosaminyltransferase (LARGE); (b) transfecting the cell with a vector that expresses alpha-DG, either prior to step (a), concurrently with step (a), or after step (a); (c) culturing the transfected cell, wherein the transfected cell secretes alpha-DG; and (d) purifying alpha-DG that is secreted from the transfected cell. In further embodiments, methods for preparing the disclosed alpha-DG may include (a) transfecting a cell that expresses like-acetylglucosaminyltransferase (LARGE) with a vector that expresses alpha-DG; (b) culturing the transfected cell, wherein the transfected cell secretes alpha-DG; and (c) purifying the alpha-DG that is secreted from the transfected cell.

### BRIEF DESCRIPTION OF THE DRAWINGS

FIG. 1. Contractile and ultrastructural properties of dystroglycan- and alpha7-deficient skeletal muscle. Specific force (A, B) and force deficit after two lengthening contractions (C, D) of the EDL muscle from alpha7-null (alpha7 KO) and DG-null (DG KO) mice were compared to those for littermate controls. Asterisks indicate significant difference (p<0.05). All data are presented as the mean±SD. E-G, Ultrastructure of quadriceps muscle from 5-week-old control (E) integrin alpha7-null (F) and DG-null (G) mice in the absence of exercise. H, Ultrastructure of exercised quadriceps muscle from DG-null mice immediately after downhill treadmill running. Black arrowheads: basal lamina; white arrowhead: sarcolemma; black asterisk: site of separation of the sarcolemma and the basal lamina; dashed line: outline of the disrupted sarcolemma; black arrow: disruption of sarcomere structure.

FIG. 2. Severe muscular dystrophy in DG/alpha7 DKO mice. (A) Total distance that the mice traveled within 12 hours in open field activity cages. (B) Vertical movement activity. Vertical movement activity was represented as the number of rearing movement. DKO significantly impaired vertical movement compared to littermates (p<0.01). The values in all data are averages from 3-7 mice of each group: WT (n=7), DG-null (n=6), alpha7-null (n=4) and DKO (n=3). (C-F) H&E staining of quadriceps sections. Severe pathological changes are observed in DKO section, including variations in fiber size, centrally located nuclei, and infiltration of inflammatory cells. White triangles: centrally nucleated cells. (G) Central nucleation is represented as the percentage of total nucleated fibers with centrally located nuclei. (H, I) Separation of the basal lamina from the sarcolemma (H) and loss of the basal lamina structure (I) in quadriceps muscles from DKO observed under electron microscopy. White arrowhead: sarcolemma; asterisk: degraded basal lamina; black arrow: detached basal lamina; white arrow: disrupted basal lamina.

FIG. 3. Characterization of the contractile properties and the DGC structure in the Large^{myd} muscle. (A) EDL muscle mass, (B) maximum force, and (C) specific force prior to subjection to the lengthening-contraction protocol. (.D) Force deficits following the lengthening-contraction protocol, as measured for EDL muscles in vitro from C57BL/6 (n=6) and Large^{myd} mice (n=6). Asterisks indicate significant difference (p<0.05). All data are presented as the mean±S.E.M. (E) Solubilized C57BL/6 and Large^{myd} skeletal muscle were enriched for DGC by WGA affinity chromatography and separated on 10-30% sucrose gradients. Gradient fractions (1=top, 13=bottom) were blotted with antibodies against core alpha-DG, dystrophin (Dys), alpha-SG, γ-SG and beta-DG. (F, G) Ultrastructural analysis of quadriceps muscles from Large^{myd} mice were observed under electron microscopy. Black arrowhead: basal lamina; white arrowhead: sarcolemma; asterisk: dissociation of basal lamina and sarcolemma.

FIG. 4. Membrane damage assay on WT and Large^{myd} skeletal muscle. (A) Schematics of the in situ membrane damage assay. Representative examples of time-lapsed images of membrane damage assay performed on C57BL/6 (B) and Large^{myd} skeletal muscle fibers in regular Tyrode buffer (C) or in a hyperosmotic buffer (D). Scale bar: 20 μm. (E) Plot of FM 1-43 fluorescence intensity against time in WT (WT, n=7) and Large^{myd} (n=8) muscle fibers. (F) Plot of FM 1-43 fluorescence intensity against time in Large^{myd} (n=5) muscle fibers in the hyperosmotic buffer. Dashed curve represents membrane damage data in Large^{myd} muscle in regular Tyrode buffer (isosmotic), from the experiment whose results are depicted in E. All data are presented as mean±S.E.M.

FIG. 5. Effect of alpha-DG-mediated association of the basal lamina with the sarcolemma on membrane integrity. (A) The purified recombinant alpha-dystroglycan reacted with the glycosylated alpha-DG antibody IIH6 (left) and bound to laminin in the laminin overlay assay (right). (B) The Large^{myd} muscles injected with recombinant alpha-DG/L (alpha-DG/L injected) or saline (Mock) were stained with IIH6 antibody. (C) Representative micrographs of membrane damage assay performed on Large^{myd} muscle fibers treated with or without recombinant alpha-DG/L. (D) Plot of FM 1-43 fluorescence intensity against time of the in situ membrane damage assay in Large^{myd} muscle fibers treated with recombinant alpha-DG/L (n=7). The dash curve represents mean FM 1-43 fluorescence intensity of the membrane damage assay in Large^{myd} muscle from the FIG. 4E. (E) Plot of FM 1-43 fluorescence intensity against time for the in situ membrane damage assay carried out in C57BL/6 muscle fibers treated with (n=9) or without LCMV (n=11). All the data are means±S.E.M.

FIG. 6. A proposed mechanism for the basement membrane-mediated prevention of membrane damage during lengthening contractions. (A) In normal skeletal muscle, the sarcolemma is tightly associated with the basement membrane. Lengthening contractions cause an increase in tension within the sarcolemma, which can lead to small membrane tears. The dysferlin-mediated membrane repair mechanism subsequently reseals the membrane and maintains membrane integrity. (B) In DG-deficient skeletal muscle, the tight association of the sarcolemma with the basal lamina is lost, and thus membrane tears developed during lengthening contractions rapidly expand, leading to loss of a large segment of the sarcolemma.

FIG. 7. Characterization of skeletal muscle dystroglycan and integrin α7 complexes. Sucrose gradient fractionation of the DGC and the integrin α7 complex from wild-type (A, C), DG-deficient (B) and integrin α7-null (D) skeletal muscle solubilized with digitonin. Glycoprotein preparations enriched by WGA-chromatography were fractionated by sucrose gradient centrifugation. Equal volumes of fractions were loaded on an SDS-PAGE gel. The blot generated from this was probed with antibodies against: integrin α7 (α7), integrin β1(β1), α-DG (α-DG), α-sarcoglycan (α-SG), β-sarcoglycan (β-SG) and γ-sarcoglycan (γ-SG). Numbers at the bottom of the blot indicate the sucrose gradient fraction number, from top to bottom.

FIG. 8. Ultrastructural analysis of quadriceps muscles from WT (A) and α7-null (B) mice after exercise. After exercise, there is no detectable abnormality in the basal lamina and the sarcolemma in the muscles from WT and α7-null mice.

FIG. 9. Exercise-induced disruption of the BL/PM complex in the DG KO muscle. (A) Immunostaining of longitudinal fibers after exercise. Five week-old mice (WT, DG KO, and α7 KO) were subjected to treadmill-exercise (15° downhill for 20 min). Immediately after the exercise, quadriceps muscles were taken. Longitudinal cryosections were immunostained with laminin and caveolin-3. Arrow: breakage of laminin- or caveolin-3-staining; arrowhead: separation of laminin- and caveolin-3-staining; asterisk: fiber with remaining laminin deposition. (B) Acute damage of DG-deficient muscle after the exercise. Cryosections of DG KO post-exercised quadriceps muscle were co-immunostained with laminin and caveolin-3. Serial section was stained with hematoxylin and eosin (H&E). White asterisk: fiber with remaining laminin deposition; black asterisk: necrotic fibers.

FIG. 10. Disrupted expression of DG and α7 in DG/α7 DKO mice. Western blotting (A) and immunofluorescence staining (B) analysis showed loss of the DG and α7 expression in DG/α7 DKO muscle. It is of note that the DG expression in DKO is higher than in the DG-null muscle due to the greater regeneration.

FIG. 11. Schematic models of the DGC in skeletal muscle of different mouse models. Left: wild-type; middle: Large^{myd}; right: MCK DG null.

FIG. 12. Immunofluorescence staining of dysferlin in skeletal muscle of Large^{myd} mice. Quadriceps muscle sections from wild-type C57BL/6 and Large^{myd} mice were stained with the anti-dysferlin antibody Hamlet.

FIG. 13. Damage assay on skeletal muscle of a 7-null mice. Plot of FM 1-43 fluorescence intensity against time in integrin α7-null (open square, n=5) muscle fibers. The dashed curve represents mean fluorescence intensity in the membrane damage assay, in wild-type muscle from FIG. 4E.

FIG. 14. Stability of recombinant α-DG on the sarcolemma of MCK-cre/Dag1^{flox/flox} muscle fibers. MCK-cre/Dag1^{flox/flox} muscles injected with recombinant α-DG/L (α-DG/L injected) or saline (Mock) were stained with the glycosylated α-DG antibody (11H6). Recombinant α-DG failed to stay on the sarcolemma of MCK-cre/Dag1^{flox/flox} muscles, suggesting that (β-DG is required for securing the recombinant α-DG on the sarcolemma.

### DETAILED DESCRIPTION

Definitions

The present invention is described herein using several definitions, as set forth below and throughout the application.

As used herein, “a,” “an,” and “the” mean “one or more” unless the context clearly dictates otherwise. For example, reference to “an alpha-dystroglycan protein” means one or more alpha-dystroglycan proteins.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” or “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.”

Disclosed herein is alpha-dystroglycan protein (alpha-DG) in glycosylated and functional form. Alpha-DG is formed from the dystroglycan precursor protein. As utilized herein, “dystroglycan” may refer to human or non-human dystroglycan. The cDNA sequence for human dystroglycan has been disclosed. (See GenBank Accession No. NM_{—}004393.2 (see also SEQ ID NO:1 and SEQ ID NO:6); and U.S. Pat. No. 5,449,616, the contents of which are incorporated herein by reference in their entireties). The polypeptide of human DG includes 895 amino acids (see SEQ ID NO:2) and is processed to release a 29 aa signal peptide from the N-terminus, the alpha-DG polypeptide (aa 30-653, see SEQ ID NO:3), and the beta-DG polypeptide (aa 654-895) from the C-terminus. (See Barresi & Campbell, J. Cell Sci., 119(2):199-207 (2006), the content of which is incorporated herein by reference in its entirety). Alpha-DG is an extracellular protein that contains three potential N-linked glycosylation sites. The mature protein has a central, highly O-glycosylated, mucin domain that connects the globular N- and C-terminal domains. (See id.). Alpha-DG may be glycosylated by like-acetylglucosaminyltransferase (LARGE). (See Barresi et al., “LARGE can functionally bypass α-dystroglycan glycosylation defects in distinct congenital muscular dystrophies,” Nat. Med. 10(7) 696-703 July 2004, the content of which is incorporated by reference in its entirety).

The presently disclosed compositions and methods may be utilized for treating or preventing diseases or disorders associated with or characterized by a dysfunctional alpha-DG. For example, diseases and disorders associated with or characterized by a dysfunctional alpha-DG may include muscular dystrophies. A “dysfunctional alpha-DG” is an alpha-DG protein or a variant or mutant thereof that exhibits an aberrant biological function or that does not exhibit its normal biological function. For example, a dysfunctional alpha-DG may not bind to one or more of the basal lamina and the sarcolemma of muscle fibers. The basal lamina includes the glycoprotein laminin, which is derived from three polypeptide chains (A, B, and C) assembled into an asymmetrical cruciform structure having three short arms and one long arm. The G domain of laminin is a large oblong globule formed by the C-terminal portion of the A chain. A dysfunctional alpha-DG as contemplated herein may not bind to laminin, and in particular, may not bind to the G domain of laminin. The sarcolemma of muscle fibers refers to the cell membrane of muscle cells. The sarcolemma includes various cell membrane glycoproteins such as beta-dystroglycan protein (beta-DG). A dysfunctional alpha-DG as contemplated herein may not bind to the sarcolemma, and in particular, may not bind to beta-DG. Methods of measuring binding activity of alpha-DG to the basal lamina and sarcolemma of muscle fibers are known in the art and are described herein. (See Examples below).

A dysfunctional alpha-DG may result from a mutation in the gene for dystroglycan (Dag1), for example, where the mutation results in an insertion, deletion, or substitution of one or more amino acids of the dystroglycan polypeptide. Alternatively, a dysfunctional alpha-DG may result from insufficient or aberrant processing of the dystroglycan precursor or the alpha-DG polypeptide. For example, a dysfunctional alpha-DG may result from insufficient processing of the dystroglycan precursor to remove the signal peptide or the beta-dystroglycan polypeptide. A dysfunctional alpha-DG also may result from insufficient glycosylation of the alpha-DG polypeptide. For example, a dysfunctional alpha-DG may result from insufficient glycosylation or the lack of glycosylation by LARGE.

A “patient in need thereof” may include a patient in need of treatment or prevention with respect to a disease or condition associated with or characterized by a dysfunctional alpha-DG. Examples of such diseases or conditions may include, but are not limited to muscular dystrophy. A “patient in need thereof” may include a patient undergoing therapy to treat muscular dystrophy. As utilized herein, muscular dystrophy (MD) refers to a group of genetic, hereditary muscle diseases characterized by progressive skeletal muscle weakness, defects in muscle proteins, and death of muscle tissue. Muscular diseases classified as muscular dystrophy include Duchenne, Becker, limb girdle, congenital, facioscapulohumeral, myotonic, oculopharyngeal, distal, Emery-Dreifuss, and over 100 other muscle diseases with similarities to muscular dystrophy. The presently disclosed compositions and methods may be utilized to treat muscular dystrophy associated with or characterized by a dysfunctional alpha-DG protein. For example, the presently disclosed compositions and methods may be utilized to treat muscular dystrophy associated with or characterized by a loss of alpha-DG from a muscular dystrophin-glycoprotein complex, for example, where the alpha-DG is dysfunctional and does not bind to at least one of the basal lamina (e.g., the G-domain of laminin) and the sarcolemma (e.g., the beta-DG protein).

As used herein, the terms “treatment,” “treat,” or “treating” refer to therapy or prophylaxis of diseases, disorders, and the symptoms thereof in a subject in need thereof. Therapy or prophylaxis typically results in beneficial or desirable clinical effects, such as alleviation of symptoms, diminishment of extent of disease, stabilization (i.e., not worsening) of the state of the disease, delay or slowing of disease progression, amelioration or palliation of the disease state, and remission (whether partial or total and, whether detectable or undetectable). “Treatment” can also mean prolonging survival as compared to expected survival if a patient were not to receive treatment. Those in need of treatment include those already with the condition or disorder as well as those prone to have the condition or disorder or those in which the condition or disorder is to be prevented.

As used herein, the term “patient” means one in need of treatment or prevention of diseases and disorders associated with or characterized by a dysfunctional alpha-DG (e.g., muscular dystrophy) or the symptoms thereof. The term “patient” may be used interchangeably herein with the term “subject” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

In the disclosed therapeutic methods, alpha-DG may be administered as part of a pharmaceutical composition. The term “pharmaceutical composition” may be utilized herein interchangeably with the term “therapeutic formulation.” Pharmaceutical compositions of alpha-DG used in accordance with the present methods may be prepared by mixing alpha-DG (which optionally is recombinant and has a desired degree of purity) together with optional pharmaceutically acceptable carriers, excipients, diluents, or stabilizers (Remington's Pharmaceutical Sciences 16th edition, Osol, A. Ed. (1980)), for example in the form of aqueous solutions or lyophilized formulations for storage. In addition to alpha-DG, the pharmaceutical compositions used in the therapeutic methods disclosed herein may contain one or more suitable pharmaceutically acceptable carriers, excipients, diluents, or stabilizers that facilitate processing of alpha-DG into preparations that can be used pharmaceutically.

A “pharmaceutically acceptable” carrier, excipient, diluent, or stabilizer typically is not biologically or otherwise undesirable, i.e., the carrier, excipient, diluent, or stabilizer may be administered to a subject, along with alpha-DG without causing any undesirable biological effects or interacting in a deleterious manner with alpha-DG or any of the other components of the pharmaceutical composition in which alpha-DG is contained. In some embodiments, the carrier, excipient, diluent, or stabilizer may be selected to minimize any degradation of alpha-DG or any of the other components of the pharmaceutical composition or to minimize any adverse side effects in the subject.

In the present methods, alpha-DG may be administered in any suitable manner. In some embodiments, alpha-DG is present in a pharmaceutical composition that has been formulated for intramuscular administration.

Suitable formulations for intramuscular administration in the methods disclosed herein include aqueous solutions of alpha-DG in water-soluble form, for example water-soluble salts. Optionally, the solution may contain stabilizers.

Formulations to be used for in vivo administration in the disclosed methods typically are sterile. Sterile compositions may be prepared, for example, by filtration through sterile filtration membranes.

The exact amount of the compositions delivered in the disclosed methods may vary from subject to subject, depending on the species, age, weight and general condition of the subject, the severity of the condition being treated, the particular composition used (e.g., with respect to concentration of alpha-DG in the composition), its mode of administration, and the like. In some embodiments, alpha-DG is administered in a dose that is effective to restore or improve membrane integrity of muscle fibers in a subject at the site at which the alpha-DG is delivered. More specifically, alpha-DG may be administered in a dose of from about 0.05 mg to about 5.0 mg per kilogram of body weight of the subject. Alpha-DG, alternatively, may be administered in a dose of from about 0.3 mg to about 3.0 mg per kilogram of body weight of the subject.

In some embodiments of the disclosed methods, alpha-DG may be administered to the patient in a dosage of between about 1 mg/ml and about 500 mg/ml. For example, alpha-DG may be administered in a dosage of about 1 mg/ml, 2 mg/ml, 3 mg/ml, 4 mg/ml, 5 mg/ml, 10 mg/ml, 15 mg/ml, 20 mg/ml, 25 mg/ml, 30 mg/ml, 35 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 105 mg/ml, 110 mg/ml, 115 mg/ml, 120 mg/ml, 125 mg/ml, 130 mg/ml, 135 mg/ml, 140 mg/ml, 145 mg/ml, 150 mg/ml, 155 mg/ml, 160 mg/ml, 165 mg/ml, 170 mg/ml, 175 mg/ml, 180 mg/ml, 185 mg/ml, 190 mg/ml, 195 mg/ml, 200 mg/ml, 205 mg/ml, 210 mg/ml, 215 mg/ml, 220 mg/ml, 225 mg/ml, 230 mg/ml, 235 mg/ml, 240 mg/ml, 245 mg/ml, 250 mg/ml, 255 mg/ml, 260 mg/ml, 265 mg/ml, 270 mg/ml, 275 mg/ml, 280 mg/ml, 285 mg/ml, 290 mg/ml, 295 mg/ml, 300 mg/ml, 305 mg/ml, 310 mg/ml, 315 mg/ml, 320 mg/ml, 325 mg/ml, 330 mg/ml, 335 mg/ml, 340 mg/ml, 345 mg/ml, 350 mg/ml, 355 mg/ml, 360 mg/ml, 365 mg/ml, 370 mg/ml, 375 mg/ml, 380 mg/ml, 385 mg/ml, 390 mg/ml, 395 mg/ml or 400 mg/ml.

In the methods, alpha-DG may be administered according to a wide variety of dosing schedules. For example, alpha-DG may be administered once or twice daily for a predetermined amount of time (e.g., four to eight weeks, or more), or according to a weekly schedule (e.g., one day per week, two days per week, three days per week, four days per week, five days per week, six days per week or seven days per week) for a predetermined amount of time (e.g., four to eight weeks, or more).

The present methods may include administering to a patient a first therapeutic agent in conjunction with a second therapeutic agent, wherein the first therapeutic agent is alpha-DG protein and the second therapeutic agent is a different therapeutic agent that is useful for a treating disease or disorder associated with or characterized by a dysfunctional alpha-protein, such as a muscular dystrophy or the symptoms thereof. By administering a first therapeutic agent “in conjunction with” a second therapeutic agent is meant that the first therapeutic agent can be administered to the patient prior to, simultaneously with, or after, administering the second therapeutic agent to the patient, such that both therapeutic agents are administered to the patient during the therapeutic regimen. For example, according to some embodiments of the present method, alpha-DG protein is administered to a patient in conjunction (i.e., before, simultaneously with, or after) administration of a second therapeutic agent for a treating disease or disorder associated with or characterized by a dysfunctional alpha-protein, or symptoms thereof. Second therapeutic agents may include agents known by the following therapeutic names: AVI-4658 (AVI Biopharma Inc.), Myodur (CepTor Corp & JCR Pharmaceuticals Inc.), FP0023 (Faust Pharmaceuticals Inc.), Biostrophin (Asklepios BioPharmaceuticals Inc.), DMD-02 (Avicena Group Inc.), MyoDys (Mirus Bio Corp & Transgene Inc.), Myo-029 (AstraZeneca Inc.), Iplex (Insmed Inc.), and CRL (CytRx Inc.).

### Illustrative Embodiments

The following Embodiments are illustrative and are not intended to limit the disclosed subject matter.

Embodiment 1. A pharmaceutical composition formulated for injection into muscle tissue and comprising alpha-dystroglycan protein, wherein the alpha-dystroglycan protein is glycosylated and binds to basal lamina and sarcolemma of muscle cells.

Embodiment 2. The composition of claim 1, wherein the alpha-dystroglycan protein is glycosylated by like-acetylglucosaminyltransferase (LARGE).

Embodiment 3. The composition of claim 1 or 2, wherein the composition comprises an effective amount of the alpha-dystroglycan protein for treating a disease or condition associated with or characterized by a dysfunctional alpha-dystroglycan protein that does not bind to at least one of basal lamina and sarcolemma of muscle cells.

Embodiment 4. The composition of any of claims 1-3 wherein the disease or condition is a muscular dystrophy associated with or characterized by loss of endogenous alpha-dystroglycan protein from a muscular dystrophin-glycoprotein complex.

Embodiment 5. The composition of any of claims 1-4, wherein the alpha-dystroglycan protein represents greater than 90% of total protein in the composition.

Embodiment 6. The composition of any of claims 1-4, wherein the alpha-dystroglycan protein represents greater than 95% of total protein in the composition.

Embodiment 7. The composition of any of claims 1-4, wherein the alpha-dystroglycan protein represents greater than 99% of total protein in the composition.

Embodiment 8. The composition of any of claims 1-7, wherein the alpha-dystroglycan protein is human alpha-dystroglycan.

Embodiment 9. The composition of any of claims 1-8, wherein the alpha-dystroglycan protein comprises SEQ ID NO:3.

Embodiment 10. The composition of any of claims 1-9, wherein the composition comprises a buffer.

Embodiment 11. The composition of any of claims 1-10. The composition of claim 1, wherein the composition is sterile and comprises 0.80-1.00% (w/v) NaCl.

Embodiment 12. The composition of any of claims 1-10, wherein the composition is sterile and comprises 0.90-0.92% (w/v) NaCl.

Embodiment 13. The composition of any of claims 1-12, comprising the alpha-dystroglycan protein at a concentration of at least about 1 mg/ml.

Embodiment 14. The composition of any of claims 1-12, comprising the alpha-dystroglycan protein at a concentration of at least about 10 mg/ml.

Embodiment 15. A method comprising injecting into muscle tissue of a patient in need thereof a pharmaceutical composition comprising alpha-dystroglycan protein, wherein the alpha-dystroglycan protein is glycosylated and binds to basal lamina and sarcolemma of muscle cells.

Embodiment 16. The method of claim 15, wherein the alpha-dystroglycan protein is glycosylated by like-acetylglucosaminyltransferase (LARGE).

Embodiment 17. The method of claim 15 or 16, wherein the patient has muscular dystrophy.

Embodiment 18. The method of claim 17, wherein the patient has a muscular dystrophy associated with or characterized by loss of endogenous alpha-dystroglycan protein from a muscular dystrophin-glycoprotein complex.

Embodiment 19. The method of any of claims 15-18, wherein the patient expresses a dysfunctional alpha-dystroglycan protein that does not bind to at least one of basal lamina and sarcolemma of muscle cells.

Embodiment 20. The method of any of claims 15-19, wherein the patient is human.

Embodiment 21. The method of any of claims 15-20, wherein the alpha-dystroglycan protein represents greater than 90% of total protein in the composition.

Embodiment 22. The method of any of claims 15-20, wherein the alpha-dystroglycan protein represents greater than 95% of total protein in the composition.

Embodiment 23. The method of any of claims 15-20, wherein the alpha-dystroglycan protein represents greater than 99% of total protein in the composition.

Embodiment 24. The method of any of claims 15-23, wherein the alpha-dystroglycan protein is human alpha-dystroglycan.

Embodiment 25. The method of any of claims 15-24, wherein the alpha-dystroglycan protein comprises SEQ ID NO:3.

Embodiment 26. The method of any of claims 15-25, wherein the composition comprises a buffer.

Embodiment 27. The method of any of claims 15-26, wherein the composition is sterile and comprises 0.80-1.00% (w/v) NaCl.

Embodiment 28. The method of any of claims 15-26, wherein the composition is sterile and comprises 0.90-0.92% (w/v) NaCl.

Embodiment 29. The method of any of claims 15-28, comprising injecting at least about 10 mg of the alpha-dystroglycan protein.

Embodiment 30. The method of any of claims 15-28, comprising injecting at least about 50 mg of the alpha-dystroglycan protein.

Embodiment 31. A composition comprising a purified alpha-dystroglycan protein, wherein the alpha-dystroglycan protein is glycosylated and binds to basal lamina and sarcolemma of muscle cells.

Embodiment 32. The composition of claim 31, wherein the purified alpha-dystroglycan protein is glycosylated by like-acetylglucosaminyltransferase (LARGE).

Embodiment 33. The composition of claim 31 or 32, wherein the purified alpha-dystroglycan protein represents greater than 90% of total protein in the composition.

Embodiment 34. The composition of claim 31 or 32, wherein the purified alpha-dystroglycan protein represents greater than 95% of total protein in the composition.

Embodiment 35. The composition of claim 31 or 32, wherein the purified alpha-dystroglycan protein represents greater than 99% of total protein in the composition.

Embodiment 36. The composition of any of claims 31-35, wherein the purified alpha-dystroglycan protein is human alpha-dystroglycan.

Embodiment 37. The composition of any of claims 31-35, wherein the purified alpha-dystroglycan protein comprises SEQ ID NO:3.

Embodiment 38. The composition of any of claims 31-37, wherein the composition comprises a buffer.

Embodiment 39. The composition of any of claims 31-38, wherein the composition is sterile and comprises 0.80-1.00% (w/v) NaCl.

Embodiment 40. The composition of any of claims 31-38, wherein the composition is sterile and comprises 0.90-0.92% (w/v) NaCl.

Embodiment 41. The composition of any of claims 31-40, comprising the purified alpha-dystroglycan protein at a concentration of at least about 1 mg/ml.

Embodiment 42. The composition of any of claims 31-40, comprising the purified alpha-dystroglycan protein at a concentration of at least about 10 mg/ml

Embodiment 43. A method for preparing a purified alpha-dystroglycan protein, the method comprising: (a) transfecting a cell with a vector that expresses alpha-dystroglycan protein; (b) transfecting the cell with a vector that expresses like-acetylglucosaminyltransferase (LARGE), either prior to step (a), concurrently with step (a), or after step (a); (c) culturing the transfected cell, wherein the transfected cell secretes alpha-dystroglycan protein; and (d) purifying the alpha-dystroglycan protein that is secreted from the transfected cell.

Embodiment 44. A method for preparing a purified alpha-dystroglycan protein, the method comprising: (a) transfecting a cell that expresses alpha-dystroglycan protein with a vector that expresses like-acetylglucosaminyltransferase (LARGE); (b) culturing the transfected cell, wherein the transfected cell secretes alpha-dystroglycan protein; and (c) purifying the alpha-dystroglycan protein that is secreted from the transfected cell.

Embodiment 45. A method for preparing a purified alpha-dystroglycan protein, the method comprising: (a) transfecting a cell with a vector that expresses like-acetylglucosaminyltransferase (LARGE); (b) transfecting the cell with a vector that expresses alpha-dystroglycan protein, either prior to step (a), concurrently with step (a), or after step (a); (c) culturing the transfected cell, wherein the transfected cell secretes alpha-dystroglycan protein; and (d) purifying the alpha-dystroglycan protein that is secreted from the transfected cell.

Embodiment 46. A method for preparing a purified alpha-dystroglycan protein, the method comprising: (a) transfecting a cell that expresses like-acetylglucosaminyltransferase (LARGE) with a vector that expresses alpha-dystroglycan protein; (b) culturing the transfected cell, wherein the transfected cell secretes alpha-dystroglycan protein; and (c) purifying the alpha-dystroglycan protein that is secreted from the transfected cell.

### EXAMPLES

The following Examples are illustrative and are not intended to limit the disclosed subject matter. Reference is made to Han R. et al, “Basal Lamina Strengthens Cell Membrane Integrity via the Laminin G Domain Binding Motif of alpha-Dystroglycan,” Proc. Nat'l. Acad. Sci. USA, published on-line, Jul. 20, 2009, hard-copy, Aug. 4, 2009, 106(31):12573-9, the content of which is incorporated herein by reference in its entirety.

Summary

Skeletal muscle basal lamina is linked to the sarcolemma through transmembrane receptors, including integrins and dystroglycan. The function of dystroglycan relies critically on posttranslational glycosylation, a common target shared by a genetically heterogeneous group of muscular dystrophies characterized by alpha-dystroglycan hypoglycosylation. Here, it is shown that both dystroglycan and integrin alpha7 contribute to force production of muscles, but that only disruption of dystroglycan causes detachment of the basal lamina from the sarcolemma and renders muscle prone to contraction-induced injury. These phenotypes of dystroglycan-null muscles are recapitulated by Large^{myd} muscles, which have an intact dystrophin-glycoprotein complex and lack only the LG domain binding motif on alpha-dystroglycan. Compromised sarcolemmal integrity is directly shown in Large^{myd} muscles and similarly in normal muscles when arenaviruses compete with matrix proteins for binding alpha-dystroglycan. These data provide direct mechanistic insight into how dystroglycan-linked basal lamina contributes to the maintenance of sarcolemmal integrity and protects muscles from damage.

Results

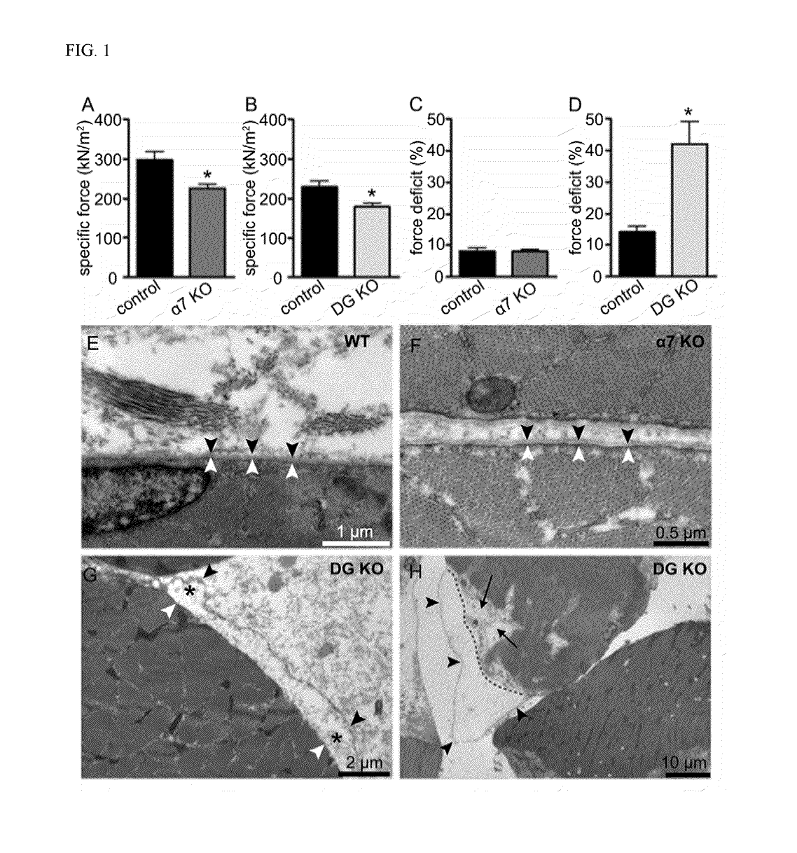

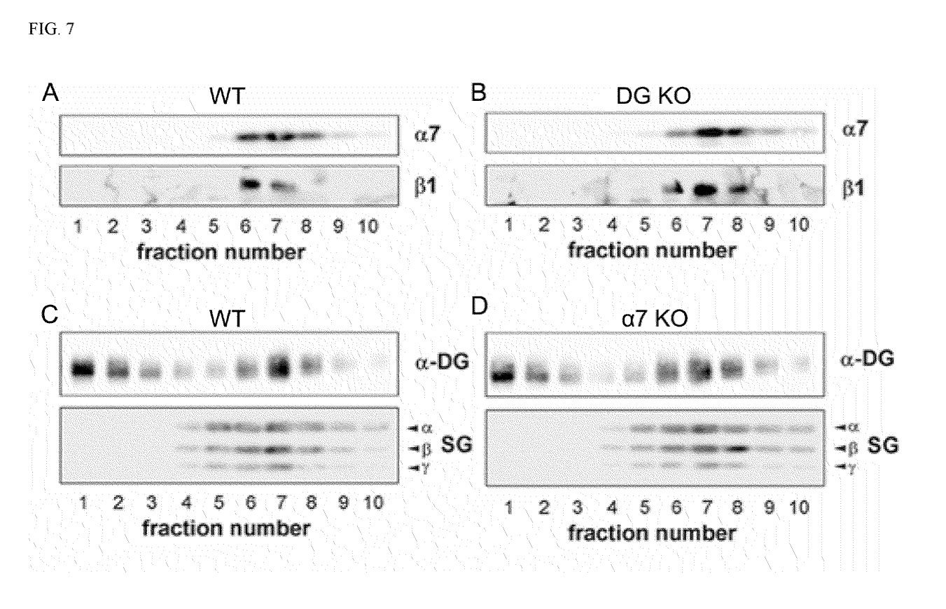

Dystroglycan and integrin play different roles in skeletal muscle. Both alpha-DG and integrins alpha7beta1 are present in skeletal muscle and function as basal lamina receptors. Using lectin affinity chromatography and sucrose gradient fractionation, DG and integrin alpha7beta1 were shown to be biochemically independent (FIG. 7). Two important features of muscular dystrophy are that the muscle produces reduced force and is more susceptible to lengthening-contraction-induced (LC-induced) damage. Thus, the roles of the basal lamina receptors (DG and integrins) on force production and force deficit in response to LC-induced muscle injury were examined by measuring the in vitro contractile properties of the extensor digitorum longus (EDL) muscles (22) of DG-deficient, integrin alpha7-null (abbreviated as alpha7-null), and wild-type (WT) mice. The specific forces (kN/m²) produced by the alpha7-null and DG-deficient EDL muscles were significantly decreased by 30% and 22%, respectively, compared to that in control muscle (FIG. 1A,B). This result indicates that both alpha7 and DG play important roles in force generation by muscle. To examine whether disruption of alpha7 and DG renders muscle more susceptible to LC-induced damage, two 30% stretches were delivered to a maximally activated EDL muscle (23) and this stretch protocol resulted in a force deficit (percentage of force loss after the stretch protocol) of around 10% in WT EDL muscle (FIG. 1C,D). The force deficit in the alpha7-null EDL muscle was not statistically different (FIG. 1C). In contrast, the force deficit of DG-deficient EDL muscle was 42%, which was 3-fold greater than that in the WT muscle (FIG. 1D). The excessive force deficit of DG-deficient muscle compared to alpha7-null and WT muscle clearly differentiates the fundamental roles of the two receptors, and demonstrates that DG plays a critical role in protecting muscle fibers from damage during lengthening contractions.

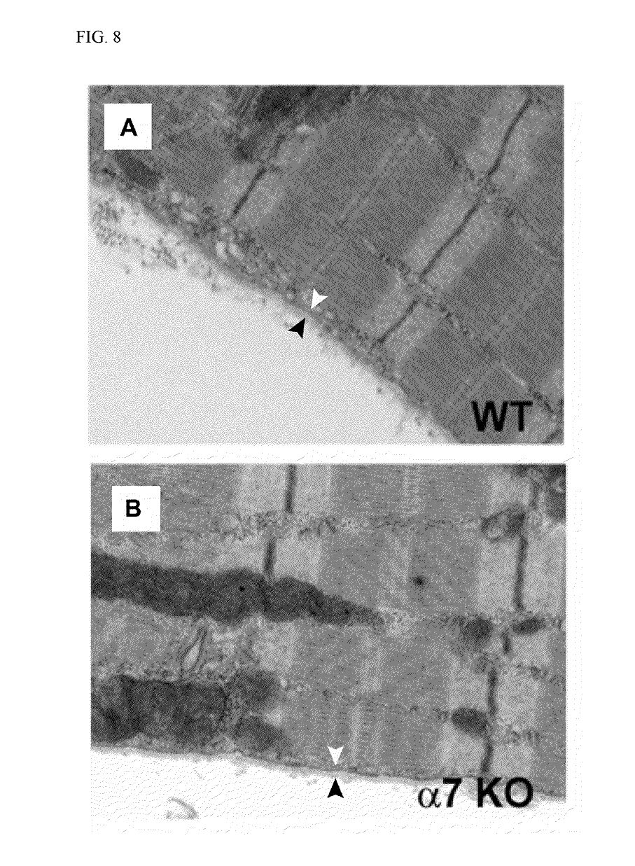

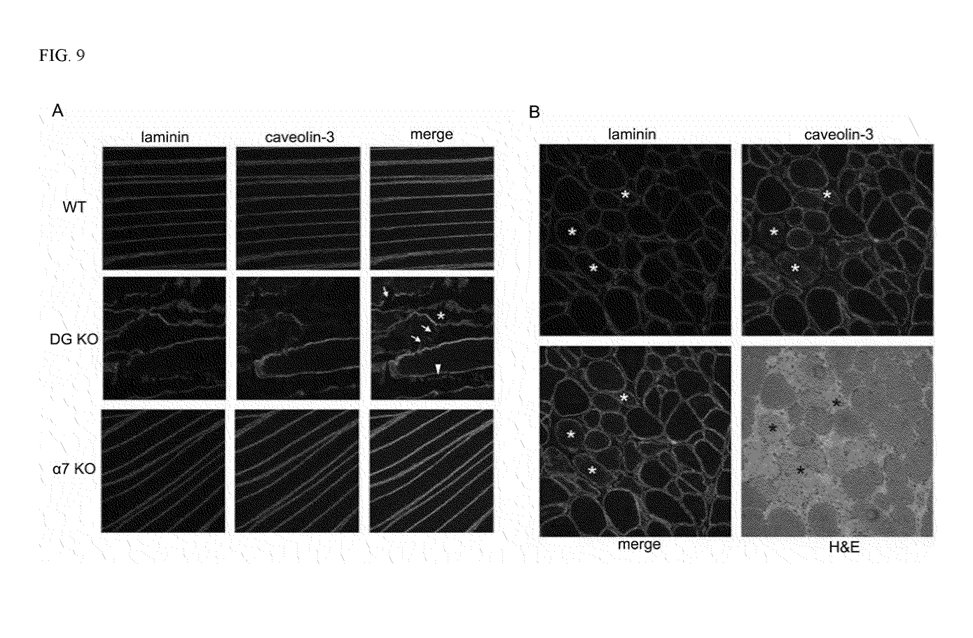

Dystroglycan is involved in anchoring the basal lamina to the sarcolemma. Since DG and integrin alpha7beta1 are basal lamina receptors in skeletal muscle, next whether the loss of DG or alpha7 causes any abnormalities in the basal lamina and/or sarcolemma of skeletal muscle was determined. Analysis of the skeletal muscle fiber ultrastructure by electron microscopy revealed that the basal lamina in both WT and alpha7-null muscle was intact, and that the association between the basal lamina and the sarcolemma was tight and continuous (FIG. 1E,F). Although DG-deficient muscle also had an intact basal lamina, an obvious separation of the basal lamina from the sarcolemma was frequently observed (FIG. 1G). The muscle ultrastructure also was analyzed after downhill treadmill exercise which causes LC-induced muscle injury in vivo. In both WT and alpha7-null muscles, no obvious changes were detected in the basal lamina, sarcolemma, and myofibril structures after the exercise (FIG. 8). However, DG-null muscle fibers showed severe detachment of the basal lamina from the rest of the fiber and disruption of the underlying sarcomere structure after the exercise (FIG. 1H and FIG. 9). These data demonstrate that DG-mediated linkage between the basal lamina and the sarcolemma may play a crucial role in the maintenance of the muscle membrane integrity during lengthening contractions.

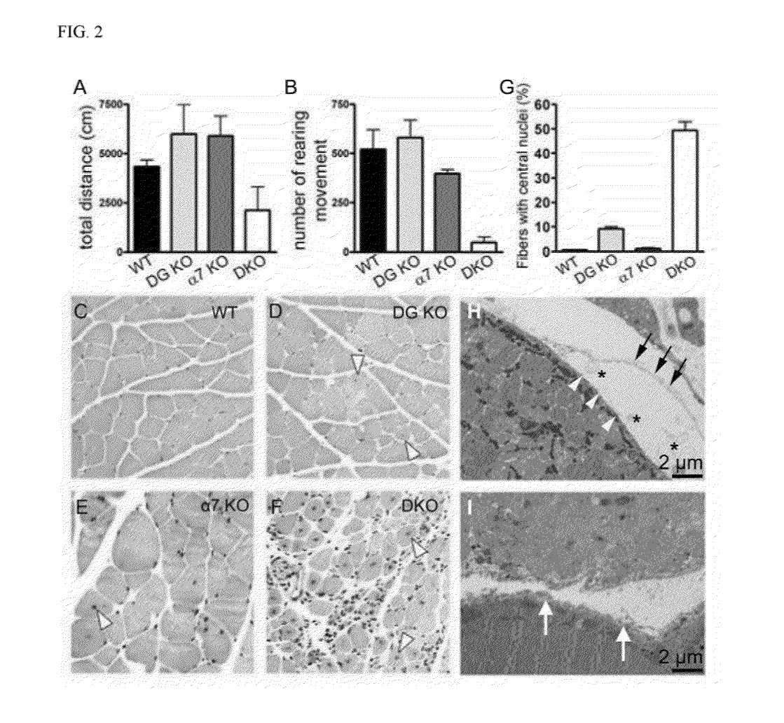

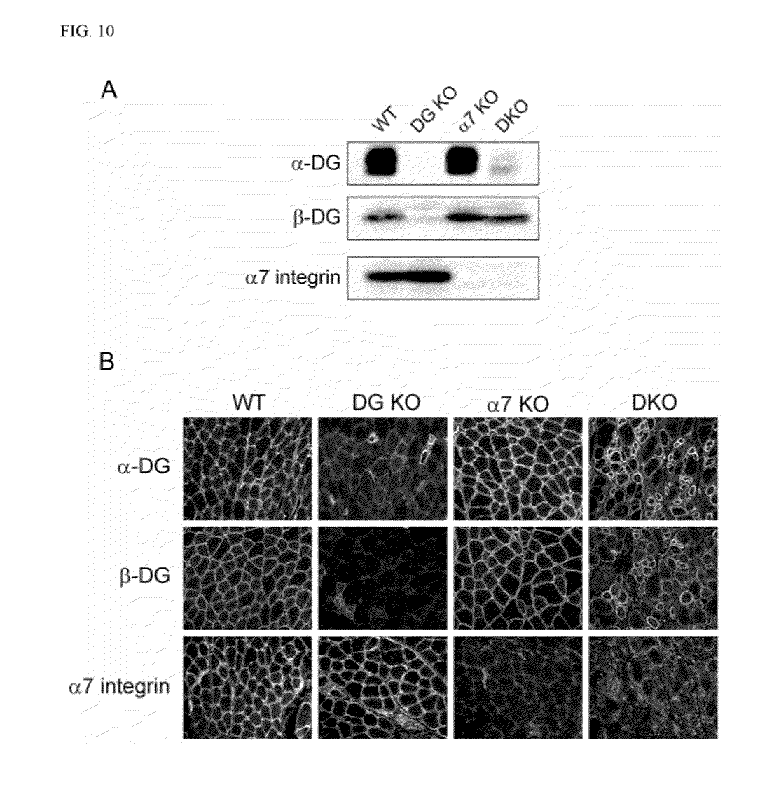

Severe muscular dystrophy in DG/alpha7 double mutant mice. Integrin and DGC show complementary expression patterns in skeletal muscle. Integrin primarily functions at the myotendinous junctions in skeletal muscle while DGC functions at both the myotendinous junctions and lateral basal lamina association (24). To further examine the functional complement of integrin and DG, DG/alpha7 double mutant (DKO) mice were created by crossing MCK-cre/Dag1^{flox/flox} and alpha7-null mice. Loss of both DG and alpha7 in quadriceps muscle of the DKO mice was confirmed by immunofluorescence and Western blotting analysis (FIG. 10). At birth, the DKO mice were indistinguishable from the littermates, but at ˜4 weeks of age the DKO mice were smaller than their littermates, and they died at ˜6-8 weeks of age. Therefore, the DKO mice and the control littermates were analyzed at 5 weeks of age. DG-null and alpha7-null mice were indistinguishable from WT mice at this age, whereas the body mass of the DKO mice were about half the mass of littermates (Table 1). Widespread decreases of muscle mass in DKO mice also were observed (Table 1). In open field activity assays, the total distance that the DKO mice traveled within 12 hours was significantly less than those traveled by either single mutant (FIG. 2A). In addition, the DKO mice showed a dramatic reduction in rearing activity, indicative of severe impairment in hind limb muscle function (FIG. 2B). Histological examination of quadriceps at 5 weeks of age revealed more severe hallmarks of muscular dystrophy in DKO mice than DG-null and alpha7-null mice, characterized by myonecrosis, central nucleation, and variation of fiber size with many small atrophic fibers (FIG. 2C-F). Moreover, infiltration of mononuclear cells was observed in the DKO skeletal muscle. At 5 weeks of age the DKO diaphragm also showed dystrophic pathology similar to the quadriceps muscle. Quantification of the number of muscle cells with central nucleation showed increases in muscle fiber regeneration in DKO compared to DG-null mice (FIG. 2G). No significant increase in central nucleation was observed in alpha7-null mice, which is consistent with the very mild phenotype in young alpha7-null mice. These data indicate more frequent on-going muscle degeneration/regeneration in the DKO muscle than each of single mutant controls. In DKO fibers, in addition to separation of the basal lamina and the sarcolemma (FIG. 2H), complete loss of the basal lamina structure was observed (FIG. 2I). To distinguish these changes from myonecrosis, disruption of the basal lamina structure was seen adjacent to normal sarcomere structure (FIG. 2I, lower fiber). Taken together, these data indicate that both DG and integrin alpha7 play essential roles in force generation and myofiber-basal lamina association.

Large^{myd} muscle maintains an intact DGC but is highly susceptible to the LC-induced force loss. The data thus far illustrated that both basal lamina receptors DG and alpha7 are important for normal skeletal muscle function, but different from alpha7, DG is required for maintaining the tight association between the sarcolemma and the basal lamina, which appears to be critical for protecting the muscle against LC-induced muscle injury. However, the DG-null muscle lacks both alpha-DG and beta-DG and thus it is possible that the increased susceptibility to LC-induced injury is caused by the loss of any intracellular connection mediated by beta-DG. To dissect out the contribution of the extracellular alpha-DG in the pathogenesis, Large^{mud} mice were utilized. Large^{myd} mice are the animal model for secondary dystroglycanopathy, which carries an intragenic deletion of exons 4-7 in the Large gene, rendering alpha-dystroglycan not properly glycosylated (25). The hypoglycosylated alpha-DG in Large^{myd} muscle lacks the important motif for binding the LG domains of many basal lamina proteins such as laminin, neurexin, agrin (14) and perlecan (26).

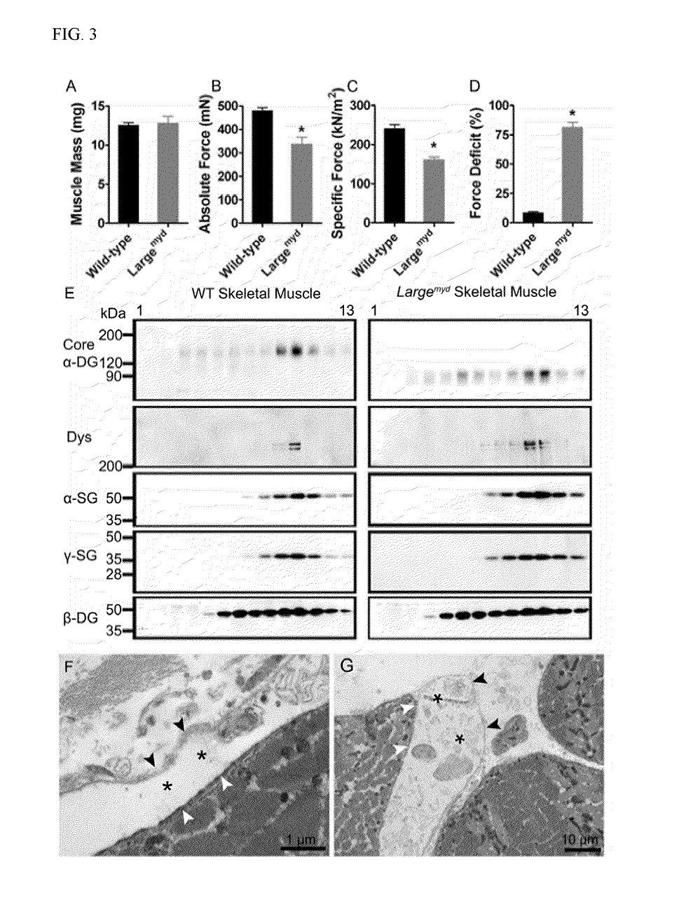

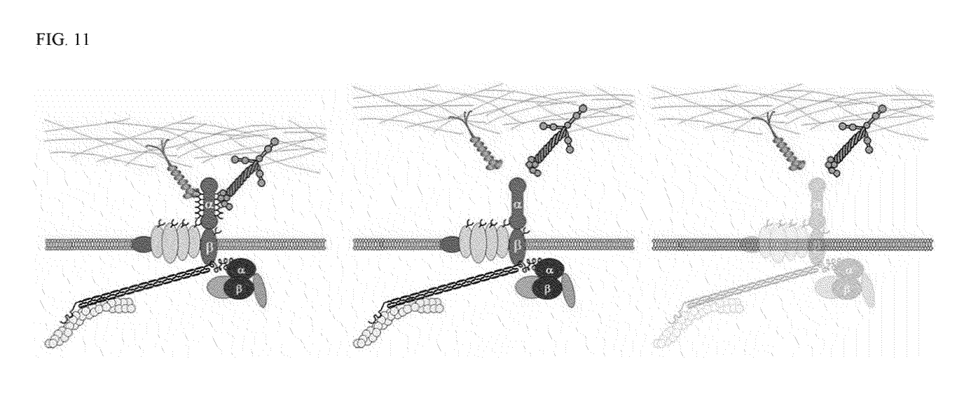

To examine whether the muscle with a glycosylation defect in alpha-DG is susceptible to LC-induced injury, contractile properties of, and force deficits in, the EDL muscles of Large^{myd} mice were measured. The mass of the Large^{myd} EDL muscle did not differ from that of the control mice (FIG. 3A), but the maximum force generated by Large^{myd} EDL muscle was 30% lower than in WT EDL muscle (FIG. 3B). Similarly, the specific force (kN/m²) of Large^{myd} muscle was decreased by 33% compared to that of WT control muscle (FIG. 3C). These data suggest that fully glycosylated alpha-DG plays an important role in the ability to transmit contraction force from the sarcomere to the basal lamina, and thus in the ability of muscle to generate force. Moreover, after two 30% stretches of a maximally activated muscle, the force deficits of Large^{myd} EDL muscle were 81% (FIG. 3D), or 10-fold greater than those in WT EDL muscle. However, using lectin affinity chromatography and sucrose gradient fractionation, the muscle of Large^{myd} mouse was observed to have an intact DGC (FIG. 3E). This is in contrast to other muscular dystrophies involving the DGC, where one primary genetic defect leads to disruption of the entire DGC, as assessed using the same assay (27, 28). This finding indicates that it is not the loss of the entire DGC, but rather the disrupted linkage between the sarcolemma and the basal lamina (due to disrupted glycosylation of alpha-DG) (FIG. 11) that is responsible for the high susceptibility to LC-induced muscle injury in secondary dystroglycanopathies.

Electron microscopy analysis of quadriceps muscles from Large^{myd} mice also showed large separation between the basal lamina and the sarcolemma (FIG. 3F,G). Such separation was also observed in muscles from dystroglycanopathy patients examined (29). Thus, detachment of the basal lamina from the sarcolemma appears to be a common feature for muscular dystrophies caused by DG dysfunction or deficiency, and is likely due to the absence of an interaction between DG and LG domain-containing extracellular matrix proteins such as laminin, agrin and perlecan.

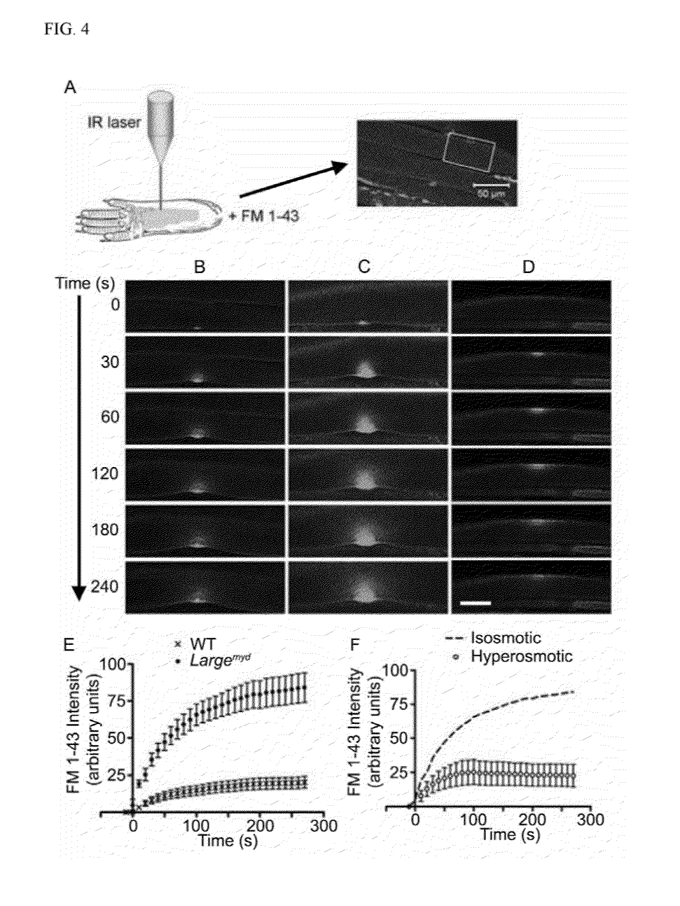

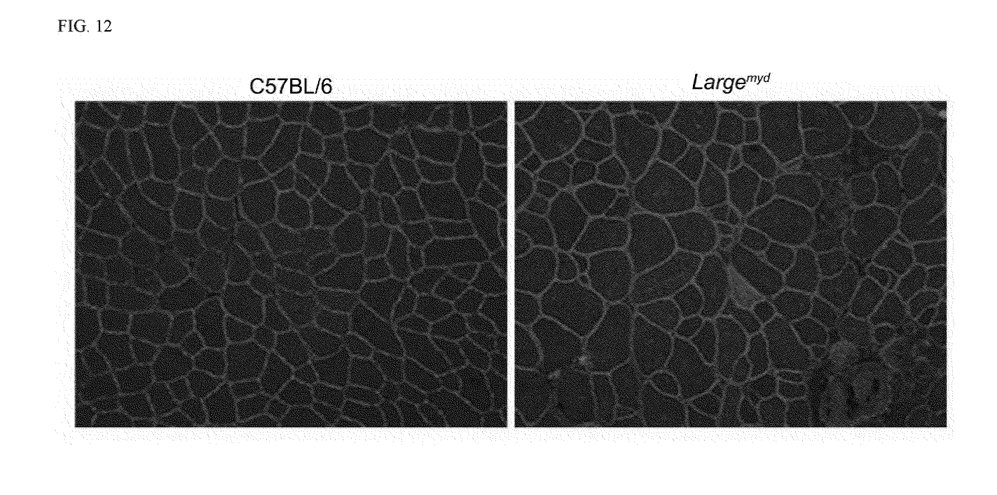

Dystroglycan deficiency compromises sarcolemma integrity. Taken together, the large force deficit following lengthening contractions (FIGS. 1 and 3), the basal lamina detachment (FIGS. 2 and 3), and the rise in serum creatine kinase activity (30) suggest that muscle is unusually susceptible to LC-induced muscle injury in the absence of functional DG, even when an intact DGC is present. This suggested that the increased susceptibility in this context is due to compromised transmission of high tensile strength (5, 6) from the basal lamina to the sarcolemma, and that this decreases sarcolemma integrity. To test this, an in situ membrane damage assay was developed (FIG. 4A). This assay uses intact muscle fibers in situ, and thus leaves the relationship between the muscle membrane and its basal lamina intact. In this assay, muscle fibers are irradiated with a mode-locked Ti-Sapphire infrared (IR) laser at 880 nm wavelength, to induce the loss of membrane integrity at a precise region of the sarcolemma in the presence of FM 1-43, a membrane-impermeant fluorescent dye. Following irradiation, the FM 1-43 fluorescence was observed to be concentrated near the laser-irradiated area, and that when the membrane integrity were restored, the increase in fluorescence accumulation halted. Accumulation of FM 1-43 fluorescence was limited to a focal region at the site of damage, and was impeded within 2 minutes in WT (FIG. 4B) muscle fibers, indicating that the membrane integrity had been restored. Large^{myd} muscle fibers subjected to the same treatment showed substantially greater FM 1-43 fluorescence accumulation (FIG. 4C) than those from WT control mice (FIG. 4B). The fluorescence intensity in both cases was plotted vs. the time post-damage (FIG. 4E) and fitted with a one-phase exponential association equation. The maximum fluorescence intensity post-irradiation based on the fitted curve was 83.6±2.9, and 20.3±1.4 (p<0.001) for Large^{myd} and WT, respectively. In contrast, the apparent rate constants did not differ significantly between the two groups (Large^{myd}, 0.016±0.002 s⁻¹; WT, 0.014±0.003 s⁻¹), suggesting that the membrane repair system is unlikely compromised in Large^{myd} muscle. Consistent with the DG-deficient muscle having a normal membrane repair system, the FM 1-43 dye did not diffuse into the entire fiber of Large^{myd} muscle as it does in dysferlin-null fibers (31-33). Also dysferlin immunostaining on the Large^{myd} muscle was normal (FIG. 12). Based on these results, it was concluded that although the potential of the membrane repair systems seems unaltered in the absence of functional DG, increased loss of the membrane integrity results in more dye entry before the membrane repair machinery can be recruited to repair it.

In further support of this, it was reasoned that reducing membrane surface tension should reduce the dye uptake in Large^{myd} muscle. Thus, the membrane damage assay in the Large^{myd} muscle was performed using a hyperosmotic buffer (normal physiological buffer supplemented with 250 mM sucrose). The muscle fiber diameters were decreased in hyperosmotic buffer (FIG. 4D), indicating that the muscle fibers shrank. Interestingly, the same level of laser irradiation resulted in very limited dye entry in hyperosmotic buffer (FIG. 4D,F). This finding further supports the conclusion that the increased dye entry observed in Large^{myd} muscle is due to an increased fragility of the sarcolemma in the absence of alpha-DG-mediated anchoring of the basal lamina to the sarcolemma.

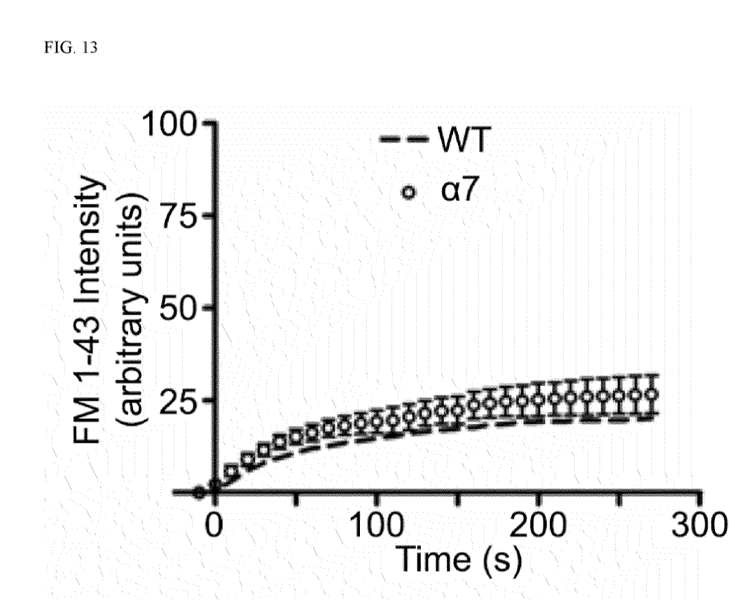

Consistent with the data showing that integrin alpha7 does not play a role in stabilizing the sarcolemma, accumulation of FM 1-43 fluorescence in integrin alpha7-null muscle fibers was similar to that in WT muscle fibers (FIG. 13).

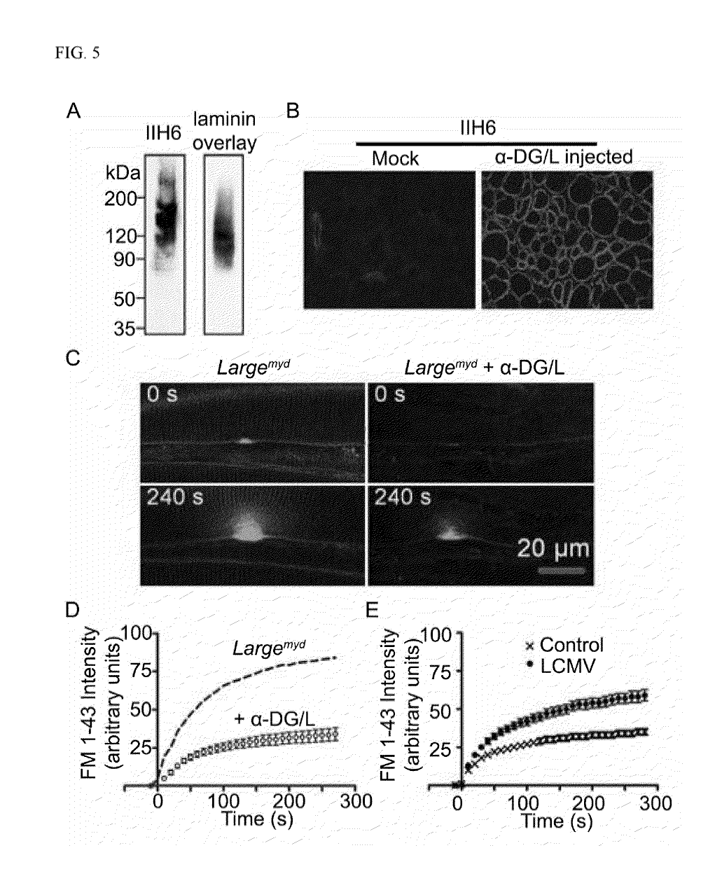

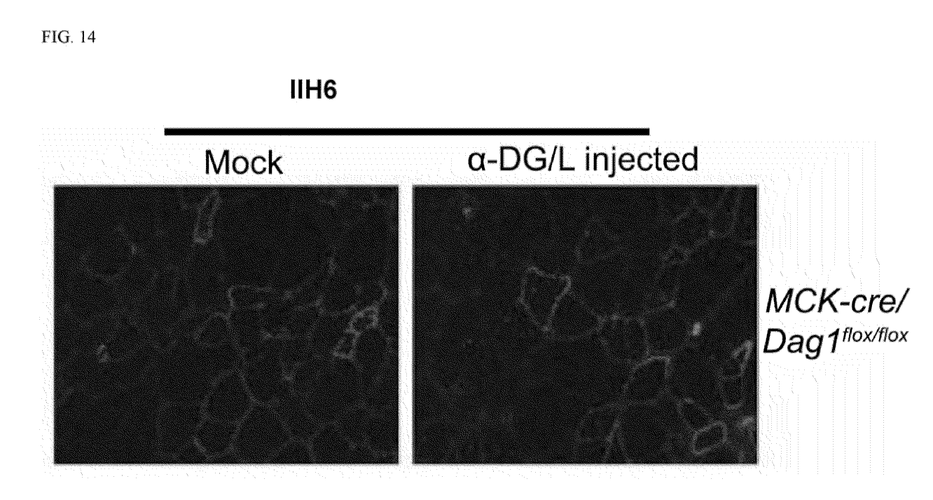

Recombinant glycosylated alpha-DG restores sarcolemma integrity in Large^{myd} muscles. Since alpha-DG is an extracellular protein, it was hypothesized that injection of recombinant alpha-DG extracellularly into Large^{myd} muscle would result in the incorporation of alpha-DG onto the muscle fibers and thus restore membrane integrity. Fully functional recombinant alpha-DG was produced in HEK293 cells that were stably co-transfected with alpha-dystroglycan and Large expression constructs and purified with lectin affinity chromatography. The purified recombinant alpha-DG had a smear appearance as the native alpha-DG from skeletal muscle on SDS-PAGE gel, was recognized by the glycosylation epitope antibody 11H6, and bound laminin in the laminin overlay assay (FIG. 5A). The purified alpha-DG then was injected into the tibialis anterior (TA) muscles in Large^{myd} mice. Immunofluorescence analysis showed that the recombinant alpha-DG successfully incorporated onto the sarcolemma (FIG. 5B). The recombinant alpha-DG also was injected into the TA muscles of MCK-cre/Dag1^{flox/flox} mice. However, the IIH6 signal was not increased compared to the non-injected muscle of the same mice (FIG. 14), suggesting that beta-DG is required for securing the recombinant alpha-DG on the sarcolemma. To examine the membrane integrity of the paw muscles from Large^{myd} mice injected with recombinant alpha-DG, the membrane damage assay was conducted on these muscle fibers. Compared to the non-injected Large^{myd} muscle fibers, the alpha-DG injected muscle fibers showed a great reduction in the dye entry after damage (FIG. 5C,D). This data suggests that the recombinant alpha-DG can bind to both the sarcolemma and the basal lamina and thereby restore normal muscle membrane integrity in Large^{myd} mouse.

Competitive LCMV-induced dissociation of the basal lamina from dystroglycan increases membrane fragility. Previously, alpha-DG was identified as a major receptor for the Old World arenavirus lymphocytic choriomeningitis virus (LCMV), as well as for the human pathogenic Lassa fever virus (LFV) (16). LCMV is able to compete with LG domain-containing basal lamina proteins for receptor binding, but unlike basal lamina proteins, the interaction between the virus and alpha-DG is not dependent on divalent cations (34). This characteristic allows examination of whether dissociation of basal lamina from alpha-DG in WT muscle in response to LCMV exposure increases susceptibility of the membrane to injury. A WT mouse hind paw preparation was incubated in Ca²⁺/Mg²⁺-free Tyrode buffer, with or without UV-inactivated LCMV clone-13 (10⁷ pfu/ml before UV inactivation). This virus preparation can bind to alpha-DG but is not infectious. The muscle preparation was then washed in normal Tyrode buffer containing Ca²⁺/Mg²⁺ and warmed to 37° C. before the membrane damage assay was performed. Pre-treatment of the muscle fibers with LCMV significantly increased the magnitude of FM 1-43 dye uptake (FIG. 5E). This result further supports the overall hypothesis that tight association of the basal lamina with the muscle sarcolemma through fully glycosylated alpha-DG strengthens the sarcolemma integrity.

Discussion

Over the course of evolution, cells have developed several strategies to maintain or recover the integrity of the plasma membrane. Previous studies have shown that animal cells can survive limited membrane insults due to an active membrane repair mechanism that involves Ca¹⁺-regulated exocytosis (32, 35). In the present study, skeletal muscle cells are shown to utilize a novel mechanism to strengthen the sarcolemma integrity—anchoring the sarcolemma to the basal lamina via laminin G domain binding motif on alpha-DG.

Secondary dystroglycanopathies are a group of severe muscular dystrophies, in which the underlying genetic defects are the genes that encode proteins known, or thought, to be important for the post-translational processing of DG (36). In contrast to the muscle fibers in other DGC-related muscular dystrophies, those in secondary dystroglycanopathies retain an intact DGC (14) but are nevertheless highly susceptible to contraction-induced injury (FIG. 3). Here, it was shown that hypoglycosylated DG fails to anchor the basal lamina to the sarcolemma, thereby rendering the muscle prone to damage. Following laser-induced membrane damage, Large^{myd} muscle fibers were shown to take up more FM 1-43 dye than WT muscle fibers. This result indicates that loss of functional alpha-DG directly renders the sarcolemma more prone to damage. This is further supported by the observations that 1) reducing membrane tension by incubating the muscle in a buffer with high osmolarity greatly reduced the dye uptake in Large^{myd} muscle fibers; 2) injection of recombinant glycosylated alpha-DG normalized the dye uptake in Large^{myd} muscle fibers; and 3) displacing the basal lamina from the sarcolemma in WT muscle fibers by adding inactivated LCMV significantly increased dye uptake.

Interestingly, the type of protection reported here seems to be conserved in other species such as yeast. Yeast and other fungi are surrounded by a cell wall, an essential structure that is required to maintain cell shape and integrity under stress. Several glycosylated proteins—including members of the WSC family (Wsc1p to Wsc4p), Mid2p and the Mid2p homologue Mtl1p—are known to play major roles in sensing the cell wall changes in yeast (39). They share a common structural organization: an extracellular domain, a transmembrane segment and a short cytoplasmic tail. The extracellular domains of these proteins are highly O- and N-glycosylated, and both types of glycosylation are essential for their functionality (39). This structure-function relationship is similar to that of DG in animals. In light of these similarities, the study here suggests that molecular transmission of the high tensile strength from an extracellular matrix to the plasma membrane is a general strategy utilized by cells to maintain the stability of their plasma membrane.

Although both DG and integrin family members function as receptors for basal lamina proteins, the data presented here clearly differentiate their primary roles in muscle fibers. The alpha7-null muscle fibers neither took up more dye in response to laser-induced membrane damage, nor were more susceptible to LC-induced muscle injury, than their WT counterparts. Furthermore, separation of the basal lamina from the sarcolemma was not observed, as in the Large^{myd} and MCK-cre/Dag1^{flox/flox} muscle fibers. The molecular basis underlying the difference between DG and integrin is unclear, but this may be related to their different binding affinity for basal lamina proteins. Integrin alpha7beta1 was reported to bind laminin only, but alpha-DG has been shown to bind a variety of basal lamina proteins containing the LG domains such as laminin (9, 40), perlecan (11) and agrin (12). In addition, considerable data showed that integrin alpha7beta1 primarily functions at the myotendinous junctions (20, 24, 41, 42) and thus by its localization its effects on lateral membrane stability may be minimal.

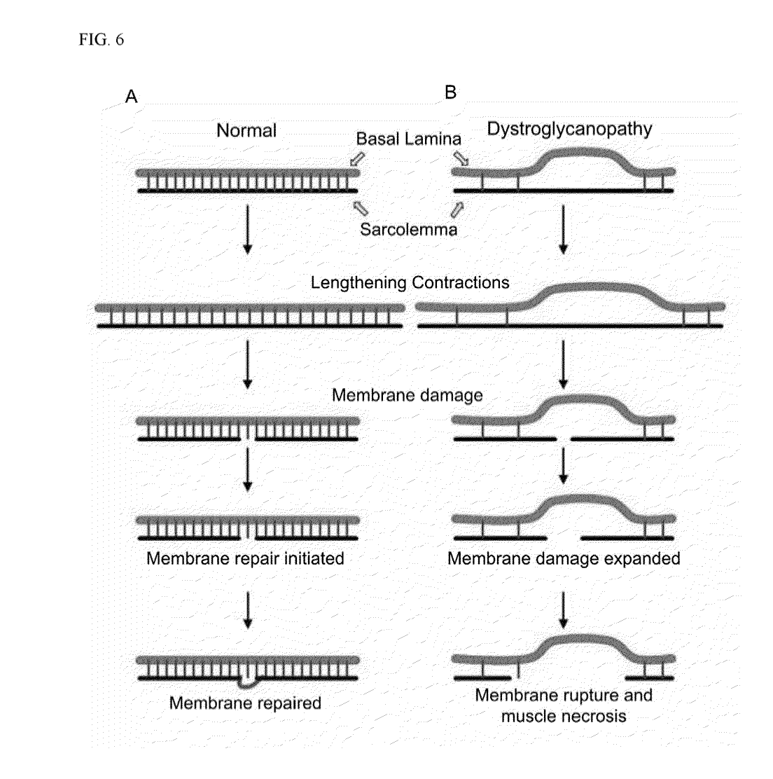

Collectively, the data here suggest that the basal lamina is tightly associated with the sarcolemma through DG binding to the LG domains of the basal lamina proteins of skeletal muscle. Lengthening contractions cause an increase in membrane tension on the sarcolemma, which can lead to small tears in the membrane. The membrane repair mechanism subsequently reseals these membrane tears and thus restores the membrane integrity of myofibers. In DG-deficient skeletal muscle, molecular linkage of the sarcolemma to the basal lamina is greatly reduced, and the tight association of the sarcolemma with the basal lamina is lost (FIG. 6). Small membrane tears caused by lengthening contractions expand, leading to the loss of a large segment of the membrane, and eventually to muscle-cell necrosis. Thus, the presence of DG allows the basal lamina (which has a much higher tensile strength than the lipid bilayer (4, 5)) to prevent the sarcolemma from rupturing. This appears to be a basic principle of fracture mechanics of thin layers or membranes: the fracture instability of a crack will not lead to further breakage if the yield stress strength of the adhesive is high enough (43). This principle of fracture mechanics can be illustrated with a balloon which fails to pop when the site of puncture is reinforced by a piece of adhesive tape. The inflated balloon represents the sarcolemma of a muscle fiber undergoing a lengthening contraction, the adhesive represents alpha-dystroglycan and the tape represents the basal lamina. The adhesive links the balloon to the tape just as alpha-dystroglycan links the sarcolemma to the basal lamina. When the tape is applied to the balloon, one can insert the needle (representative of a membrane tear) through the tape and the balloon without rupturing the balloon. In this case the presence of the tape, which has a much higher tensile strength than the balloon, prevents rapid crack advance and thus rupture. In the absence of the tape or adhesive, the balloon does not have enough stress strength, thus the needle ruptures the balloon. Therefore, DG-dependent tight physical attachment of the basal lamina to the sarcolemma is important for transmission of the basal lamina's structural strength to the sarcolemma in order to provide resistance to mechanical stress. The findings here support the idea that reinforcement of the basal lamina/sarcolemma attachment is a basic cellular mechanism that allows cell survival in tissues subjected to mechanical stress.

Materials and Methods

Measurement of contractile properties and analysis of muscle membrane structure. Mice (Large^{myd}, MCK-cre/Dag1^{flox/flox}, integrin alpha7-null, and WT littermate control mice) were maintained at The University of Iowa Animal Care Unit in accordance with animal use guidelines. All animal studies were authorized by the Animal Care Use and Review Committee of The University of Iowa. Muscle mass, fiber length, and maximum force were measured on six EDL muscles from 6- to 7-month-old aforementioned mice except Large^{myd} mice (3-5-month-old were used). Total cross-sectional area (CSA, cm) and specific Pₒ (kN/m²) were determined (22). The susceptibility of muscles to contraction-induced injury was assessed by two lengthening contractions with a strain of 30% of fiber length (23). The differences between the experimental and WT samples were assessed by a one-tailed Student's t-test, with the assumption of two-sample equal variance. Quadriceps muscles from non-exercised and exercised mice were prepared for examination by electron microscopy or immunofluorescence as described below. Lectin affinity chromatography and sucrose gradient fractionation were used to analyze the membrane protein complex integrity as described below.

Membrane damage assay. The membrane damage assay was performed on skeletal muscle fibers of 6-8 week-old mice from Large^{myd} integrin alpha7-null, and WT littermate control mice. The whole foot was cut off and the skin was removed. The connective tissues and blood vessels were trimmed off to completely expose the muscle fibers. This preparation was placed in a glass-bottom culture dish filled with Tyrode solution containing 1.8 mM Ca²⁺. Individual fibers were selected for the assay. Membrane damage was induced in the presence of 2.5 μM FM 1-43 dye (Molecular Probes) with a two-photon confocal laser-scanning microscope (LSM 510; Zeiss) coupled to a 10-W Argon/Ti:sapphire laser. After scanning of images pre-damage, a 7.9 μm×4.4 μm area of the sarcolemma on the surface of the muscle fibre was irradiated at full power for 1.29 seconds. Fluorescence images were captured at 10 second intervals for 10 min. after the initial damage. The fluorescence intensities at the damaged site were semiquantified using ImageJ software. To test the effect of reduced membrane tension on membrane integrity, the assay was also performed on Large^{myd} fibers when placed in a hyperosmotic solution as discussed below. The effects of the UV-inactivated LCMV clone 13 (10⁷ pfu/ml) and recombinant glycosylated alpha-DG (see methods described below) on membrane integrity in WT and Large^{hyd} muscle fibers, respectively, were also examined using this assay.

Mice. Mice with striated-muscle specific DG deficiency (MCK-cre/Dag1^{flox/flox}) (1) and integrin α7-null (2) mice were described previously. For a direct comparison of DG-deficient and integrin α7-null skeletal muscle in the same mouse line, these two mouse lines were crossed to one another. MCK-Cre male mice bearing the floxed dystroglycan allele were mated to integrin α7 heterozygous females. F1 and F2 offspring were mated to produce F2- and F3-generation mice, respectively. Identification of the mutant mice was performed by PCR genotyping of genomic DNA prepared from mouse tail snips. The Large^{myd} colony was originally obtained from Jackson Laboratories. Mice were maintained at The University of Iowa Animal Care Unit in accordance with animal use guidelines. All animal studies were authorized by the Animal Care Use and Review Committee of The University of Iowa. For treadmill exercise, mice (˜5 week-old) were placed on an endless conveyor-type belt with a shock grid at the end (AccuPacer Treadmill, AccuScan Instruments, Columbus, Ohio) and exercised on a down-hill grade at 15 m/min for 20 min. Immediately after the exercise, mice were euthanized and quadriceps muscles were prepared for examination by electron microscopy or immunofluorescence.

Lectin affinity chromatography and sucrose gradient fractionation. Total muscle homogenates in TBS (50 mM Tris-Cl pH 7.4, 150 mM NaCl) were solubilized with 1% digitonin. After centrifugation at 140,000×g for 37 min, solubilized proteins in the supernatant were mixed with wheat germ agglutinin (WGA)-agarose beads (Vector Laboratories) and rotated end-over-end at 4° C. for 2 hours. WGA-bound proteins were eluted with TBS containing 0.3 M N-acetyl-D-glucosamine and 0.1% digitonin. The eluant was applied to a 5-30% sucrose gradient and centrifuged at 215,000×g for 90 min. Fractions (1 ml) were collected from the top of the gradient and analyzed by SDS-PAGE.

Measurement of contractile properties. Muscle mass, fiber length, and maximum force were measured on 6 EDL muscles from 6- to 7-month-old Large^{myd}, MCK-cre/Dag1^{flox/flox}, integrin α7-null, and wild-type littermate control mice. Mice were anesthetized and muscles isolated and stimulated to provide maximum isometric tetanic force (Pₒ). The susceptibility of muscles to contraction-induced injury was assessed by two lengthening contractions with a strain of 30% of fiber length. Total cross-sectional area (CSA, cm²) and specific Pₒ (kN/m²) were determined (3). The differences between the experimental and wild-type samples were assessed by a one-tailed Student's t-test, with the assumption of two-sample equal variance.

Mouse behavior analysis. Locomotor activity was monitored using Digiscan Animal Activity Monitoring System running Versamax Windows software (Accuscan Instruments, Columbus, Ohio). The Versamax Windows software uses a mathematical algorithm to compute total distance traveled (in cm) and rearing number. All mice were tested for 12 hours starting from 6 pm.

Membrane damage assay. The membrane damage assay was performed on skeletal muscle fibers of 6-8 week-old mice from Large^{myd}, integrin α7-null, and wild-type littermate control mice. The whole foot was cut off and the skin was removed. The connective tissues and blood vessels were trimmed off to completely expose the muscle fibers. This preparation was placed in a glass-bottom culture dish filled with Tyrode solution containing 1.8 mM Ca²⁺. Individual fibers were selected for the assay. Regenerating muscle fibers (centrally-nucleated or with small diameters) were carefully excluded from the assay. Membrane damage was induced in the presence of 2.5 μM FM 1-43 dye (Molecular Probes) with a two-photon confocal laser-scanning microscope (LSM 510; Zeiss) coupled to a 10-W Argon/Ti:sapphire laser. After scanning of images pre-damage, a 7.9 μm×4.4 μm area of the sarcolemma on the surface of the muscle fibre was irradiated at full power for 1.29 seconds. Fluorescence images were captured at 10 second intervals for 10 min. after the initial damage. The fluorescence intensities at the damaged site were semiquantified using ImageJ software.

Production of recombinant glycosylated α-DG. Stable HEK293F cell lines (Invitrogen) expressing both of α-dystroglycan and Large were generated to produce the recombinant α-dystroglycan that bound LG domain proteins with high affinity. An expression vector, named pcDNA3_aDG, was made by insertion of partial rabbit DAG1 cDNA into pcDNA3. (See SEQ ID NO:4). A similar expression vector, named pcDNA3_haDG, was made by insertion of partial human DAG1 cDNA into pcDNA3. (See SEQ ID NO:6). The insert DNA of pcDNA3_aDG and pcDNA3_haDG encode the entire rabbit alpha-DG and human alpha-DG, respectively, but not the beta-DG polypeptide region. pPuro-LARGE, which was used to express LARGE, was made by insertion of human LARGE cDNA with an in-frame addition of the 6× His coding sequence at the 3′ end into ORES puro 3 (Clontech). (See SEQ ID NO:5).

HEK293F (Invitrogen) was transfected with pcDNA3_aDG or pcDNA3_haDG using Fugene6 (Gibco). Post-transfection 48 hours, the cells were cultivated with 10% FBS-DMEM media supplemented with glutamate, penicillin and streptomycin in addition to G418, which is the resistant marker of pcDNA3. Single cells which have resistance to G418 were isolated manually and allowed to expand in 48-wells culture plate. Excreted recombinant alpha-DG in the media was enriched by agarose-bound Wheat Germ Agglutinin (Vector laboratories) and tested by Immunoblotting with anti-Dystroglycan antibody. Cells expressing alpha-DG strongly were selected as stable cell lines and named HEK293-aDG or HEK293-haDG, respectively.

HEK293-aDG or HEK293-haDG were further transfected with pPuro-LARGE using Fugene6 (Gibco). Transfected cells were selected based on the resistance against puromycin, which is the resistant marker of pPuro-LARGE, as described above. Excreted recombinant alpha-DG in the media was enriched by agarose-bound Wheat Germ Agglutinin (Vector laboratories) and tested by Immunoblotting with IIH6, which recognizes laminin-binding form alpha-DG. Cells expressing alpha-DG, which has high immunoreactivity against this antibody, were selected as stable cell lines and named HEK293-aDG/L or HEK293-haDG/L, respectively.

Injection of purified recombinant α-DG into Large^{myd} muscles. Prior to the injection to Large^{myd} mice, the buffer was changed to sterile 0.9% saline by Amicon Ultra (Millipore). The calf, tibial anterior, and paw muscles of Large^{myd} mice were injected with 50, 30, and 10 μl of the purified recombinant rabbit α-dystroglycan (200 μg/ml) or saline, respectively. The muscles were excised five days post injection and were analyzed by immunofluorescence staining or membrane damage assay.

Laminin overlay assay. Laminin overlay assays were performed on PVDF membranes using mouse Engelbreth-Holm-Swarm (EHS) laminin as previously described (4). Briefly, PVDF membranes were blocked in laminin-binding buffer (LBB: 10 mM triethanolamine, 140 mM NaCl, 1 MM MgCl₂, 1 mM CaCl₂, pH 7.6) containing 5% BSA followed by incubation with laminin overnight at 4° C. in LBB. Membranes were washed and incubated with anti-laminin (Sigma) followed by anti-rabbit IgG-HRP. Blots were developed by enhanced chemiluminescence.

LCMV treatment of wild-type muscle. The wild-type mouse foot preparation was incubated with or without the UV-inactivated LCMV clone 13 (10⁷ pfu/ml) in ice-cold Ca⁷⁺/Mg²⁺-free Tyrode solution for two hours. The preparation was then washed twice with ice-cold normal Ca²⁺/Mg²⁺-containing Tyrode solution, and warmed up to 37° C. The membrane damage assay was then conducted on these samples as described above.

Electron microscopy. Mice were anesthetized with ketamine (87.5 mg/kg body weight), and a bilateral sternum incision was performed to expose the left atrium. Mice were perfused with PBS and then with 2% paraformaldehyde in PBS. Quadriceps muscle blocks were dissected into pieces (1 mm×3 mm) and fixed using Karnowsky's fixative (2.5% glutaraldehyde and 2% paraformaldehyde in 0.1 M cacodylate buffer, pH 7.4) for 2 hours at 4° C. Tissue blocks were washed in 0.1 M cacodylate buffer (2×5 min), processed through a 6-hour routine EM processing schedule, and then infiltrated with epon/alardite resin (Electron Microscopy Sciences, Fort Washington, Pa.) on a Leica EM TP automatic tissue processor. Tissues were embedded, oriented longitudinally and transversely, placed in a vacuum-infiltrating oven, and then polymerized at 60° C. for 24 hours. Multiple 1-micron thick sections were stained with 1% toluidine blue in 1% borax. Representative areas were selected, ultrasectioned at 70 nm (silver sections), mounted on 200 mesh athene copper grids, double stained with Reynolds lead citrate and uranyl acetate, and then examined using a Zeiss 906E electron microscope. Representative digital images were taken using SIS Keenview camera and software.

TABLE 1
Severe loss of body weight and muscle mass in DG/α7 DKO mice.

| | Cont. | DG KO | α7 KO | double KO |
|---|---|---|---|---|
| Body weight | 23.6 ± 0.4 | 21.7 ± 3.1 | 20.8 ± 1.0* | 11.2 ± 0.9*** |
| (g) | | | | |
| Gastroc- | 121.4 ± 5.5 | 125.1 ± 8.3 | 122.8 ± 8.2 | 37.5 ± 0.6*** |
| nemius (mg) | | | | |
| TA (mg) | 34.9 ± 5.5 | 33.5 ± 1.5 | 35.0 ± 1.6 | 10.1 ± 2.4*** |
| Triceps (mg) | 72.0 ± 11.6 | 74.0 ± 21.3 | 62.3 ± 2.1 | 22.0 ± 3.2*** |
| Quad. (mg) | 107.4 ± 2.5 | 126.3 ± 12.5 | 116.5 ± 17.6 | 40.6 ± 9.6*** |
| Heart (mg) | 115.1 ± 14 | 128.5 ± 34.2 | 113.9 ± 19.5 | 71.9 ± 4.8** |
| Shin length | 2.2 ± 0 | 2.2 ± 0.1 | 2.1 ± 0.1 | 2.1 ± 0.1 |
| (cm) | | | | |

*p < 0.05;
**p < 0.01;
***p < 0.001; n = 3

### REFERENCES

1. Davies K E, Nowak K J (2006) Molecular mechanisms of muscular dystrophies: old and new players. Nat Rev Mol Cell Biol 7:762-773.

2. Durbeej M, Campbell K P (2002) Muscular dystrophies involving the dystrophin-glycoprotein complex: an overview of current mouse models. Curr Opin Genet Dev 12:349-361.

3. Meryon E (1852) On granular and fatty degeneration of voluntary muscles. Med Chir Trans 35:73-84.

4. Sanes J R (2003) The basement membrane/basal lamina of skeletal muscle. J Biol Chem 278:12601-12604.

5. Candiello J, et al. (2007) Biomechanical properties of native basement membranes. FEBS J 274:2897-2908.

6. Grounds M D Sorokin L, White J (2005) Strength at the extracellular matrix-muscle interface. Scand J Med Sci Sports 15:381-391.

7. Timpl R, Brown J C (1996) Supramolecular assembly of basement membranes. Bioessays 18:123-132.

8. Miner J H, Yurchenco A D (2004) Laminin functions in tissue morphogenesis. Annu Rev Cell Dev Biol 20:255-284.

9. Ibraghimov-Beskrovnaya 0, et al. (1992) Primary structure of dystrophin-associated glycoproteins linking dystrophin to the extracellular matrix. Nature 355:696-702.

10. Burkin D J, Kaufman S J (1999) The alpha7beta1 integrin in muscle development and disease. Cell Tissue Res 296:183-190.

11. Talts J F, Andac Z, Gohring W, Brancaccio A, Timpl R (1999) Binding of the G domains of laminin alpha1 and alpha2 chains and perlecan to heparin, sulfatides, alpha-dystroglycan and several extracellular matrix proteins. EMBO J 18:863-870.

12. Gee S H, Montanaro F, Lindenbaum M H, Carbonetto S (1994) Dystroglycan-alpha, a dystrophin-associated glycoprotein, is a functional agrin receptor. Cell 77:675-686.

13. Michele D E, Campbell K P (2003) Dystrophin-glycoprotein complex: post-translational processing and dystroglycan function. J Biol Chem 278:15457-15460.

14. Michele D E, et al. (2002) Post-translational disruption of dystroglycan-ligand interactions in congenital muscular dystrophies. Nature 418:417-422.

15. Barresi R, et al. (2004) LARGE can functionally bypass alpha-dystroglycan_slycosylation defects in distinct congenital muscular dystrophies. Nat Med 10:696-703.

16. Cao W, et al. (1998) Identification of alpha-dystroglycan as a receptor for lymphocytic choriomeningitis virus and Lassa fever virus. Science 282:2079-2081.

17. Rambukkana A, et al. (1998) Role of alpha-dystroglycan as a Schwann cell receptor for Mycobacterium leprae. Science 282:2076-2079.

18. Williamson R A, et al. (1997) Dystroglycan is essential for early embryonic development: disruption of Reichert's membrane in Dag1-null mice. Hum Mol Genet 6:831-841.

19. Mayer U (2003) integrins: redundant or important players in skeletal muscle? J Biol Chem 278:14587-14590.

20. Mayer U, et al. (1997) Absence of integrin alpha 7 causes a novel form of muscular dystrophy. Nat Genet 17:318-323.

21. Hayashi Y K, et al. (1998) Mutations in the integrin alpha7 gene cause congenital myopathy. Nat Genet 19:94-97.

22. Brooks S V, Faulkner J A (1988) Contractile properties of skeletal muscles from young, adult and aged mice. J Physiol 404:71-82.

23. Dellorusso C, Crawford R W, Chamberlain J S, Brooks S V (2001) Tibialis anterior muscles in mdx mice are highly susceptible to contraction-induced injury. J Muscle Res Cell Motil 22:467-475.

24. Kaariainen M, et al. (2000) Integrin and dystrophin associated adhesion protein complexes during regeneration of shearing-type muscle injury. Neuromuscul Disord 10:121-132.

25. Grewal P K, Holzfeind P J, Bittner R E, Hewitt J E (2001) Mutant glycosyltransferase and altered glycosylation of alpha-dystroglycan in the myodystrophy mouse. Nat Genet 28:151-154.

26. Kanagawa M, et al. (2005) Disruption of perlecan binding and matrix assembly by post-translational or genetic disruption of dystroglycan function. FEBS Lett 579:4792-4796.

27. Durbeej M, et al. (2000) Disruption of the beta-sarcoglycan gene reveals pathogenetic complexity of limb-girdle muscular dystrophy type 2E. Mol Cell 5:141-151.

28. Beedle A M, Nienaber P M, Campbell K P (2007) Fukutin-related protein associates with⁻the sarcolemmal dystrophin-glycoprotein complex. J Biol Chem 282:16713-16717.

29. Sabatelli P, et al. (2003) Extracellular matrix and nuclear abnormalities in skeletal muscle of a patient with Walker-Warburg syndrome caused by POMT1 mutation. Biochim Biophys Acta 1638:57-62.

30. Cohn R D, et al. (2002) Disruption of DAG1 in differentiated skeletal muscle reveals a role for dystroglycan in muscle regeneration. Cell 110:639-648.

31. Bansal D, et al. (2003) Defective membrane repair in dysferlin-deficient muscular dystrophy. Nature 423:168-172.

32. Han R, Campbell K P (2007) Dysferlin and muscle membrane repair. Curr Opin Cell Biol 19:409-416.

33. Han R, et al. (2007) Dysferlin-mediated membrane repair protects the heart from stress-induced left ventricular injury. J Clin Invest 117:1805-1813.

34. Kunz S, Sevilla N, McGavern D B, Campbell K P, Oldstone M B (2001) Molecular analysis of the interaction of LCMV with its cellular receptor [alpha]-dystroglycan. J Cell Biol 155:301-310.

35. McNeil P L, Kirchhausen T (2005) An emergency response team for membrane repair. Nat Rev Mol Cell Biol 6:499-505.

36. Muntoni F, Torelli S, Brockington M (2008) Muscular dystrophies due to glycosylation defects. Neurotherapeutics 5:627-632.

37. Matsumura K, Ervasti J M, Ohlendieck K, Kahl S D, Campbell K P (1992) Association of dystrophin-related protein with dystrophin-associated proteins in mdx mouse muscle. Nature 360:588-591.

38. Kanagawa M, et al. (2009) Residual laminin-binding activity and enhanced dystroglycan glycosylation by LARGE in novel model mice to dystroglycanopathy. Hum Mol Genet 18:621-631.

39. Levin D E (2005) Cell wall integrity signaling in Saccharomyces cerevisiae. Microbiol Mol Biol Rev 69:262-291.

40. Ervasti J M, Campbell K P (1993) A role for the dystrophin-glycoprotein complex as a transmembrane linker between laminin and actin. J Cell Biol 122:809-823.

41. van der Flier A, et al (1997) Spatial and temporal expression of the beta 1D integrin during mouse development. Dev Dyn 210:472-486.

42. Miosge N, Klenczar C, Herken R, Willem M, Mayer U (1999) Organization of the myotendinous junction is dependent on the presence of alpha7beta1 integrin. Lab Invest 79:1591-1599.

43. Broek D (1986) Elementary Engineering Fracture Mechanics (Martinus Nijhoff, Dordrecht) 4th Ed.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A pharmaceutical composition formulated for injection into muscle tissue and comprising alpha-dystroglycan protein, wherein the alpha-dystroglycan protein is glycosylated and binds to basal lamina and sarcolemma of muscle cells.

2. The composition of claim 1, wherein the alpha-dystroglycan protein is glycosylated by like-acetylglucosaminyltransferase (LARGE).

3. The composition of claim 1, wherein the composition comprises an effective amount of the alpha-dystroglycan protein for treating a disease or condition associated with or characterized by a dysfunctional alpha-dystroglycan protein that does not bind to at least one of basal lamina and sarcolemma of muscle cells.

4. The composition of claim 3, wherein the disease or condition is a muscular dystrophy associated with or characterized by loss of endogenous alpha-dystroglycan protein from a muscular dystrophin-glycoprotein complex.

5. The composition of claim 1, wherein the alpha-dystroglycan protein represents greater than 90% of total protein in the composition.

6. The composition of claim 1, wherein the alpha-dystroglycan protein is human alpha-dystroglycan.

7. The composition of claim 1, wherein the alpha-dystroglycan protein comprises SEQ ID NO:3.

9. The composition of claim 1, wherein the composition is sterile and comprises 0.80-1.00% (w/v) NaCl.

10. The composition of claim 1, comprising the alpha-dystroglycan protein at a concentration of at least about 1 mg/ml.

11. A method comprising injecting into muscle tissue of a patient in need thereof the pharmaceutical composition of claim 1.

12. The method of claim 11, wherein the patient has muscular dystrophy.

13. The method of claim 11, wherein the patient has a muscular dystrophy associated with or characterized by loss of endogenous alpha-dystroglycan protein from a muscular dystrophin-glycoprotein complex.

14. The method of claim 11, wherein the patient expresses a dysfunctional alpha-dystroglycan protein that does not bind to at least one of basal lamina and sarcolemma of muscle cells.

15. The method of claim 11, wherein the patient is human.

16. A composition comprising a purified alpha-dystroglycan protein, wherein the alpha-dystroglycan protein is glycosylated and binds to basal lamina and sarcolemma of muscle cells.

17. A method for preparing a purified alpha-dystroglycan protein, the method comprising:
(a) transfecting a cell with one or more vectors that express alpha-dystroglycan protein, like-acetylglucosaminyltransferase (LARGE), or both;
(b) culturing the transfected cell, wherein the transfected cell secretes glycosylated alpha-dystroglycan protein; and
(d) purifying the alpha-dystroglycan protein that is secreted from the transfected cell.

18. The method of claim 17, wherein step (a) comprises: (i) transfecting a cell with a vector that expresses alpha-dystroglycan protein; and (ii) transfecting the cell with a vector that expresses like-acetylglucosaminyltransferase (LARGE), either prior to step (a.i.), concurrently with step (a.i.), or after step (a.i.).

19. The method of claim 17, wherein step (a) comprises: (i) transfecting a cell with a vector that expresses like-acetylglucosaminyltransferase (LARGE); and (ii) transfecting the cell with a vector that expresses alpha-dystroglycan protein, either prior to step (a.i.), concurrently with step (a.i.), or after step (a.i.).

20. The method of claim 17, wherein step (a) comprises transfecting a cell that expresses alpha-dystroglycan protein with a vector that expresses like-acetylglucosaminyltransferase (LARGE).

21. The method of claim 17, wherein step (a) comprises transfecting a cell that expresses like-acetylglucosaminyltransferase (LARGE) with a vector that expresses alpha-dystroglycan protein.
